(19) United States
(12) Patent Application Publication
(10) Pub. No.: US 2010/0185146 A1
(43) Pub. Date: Jul. 22, 2010

(54) DRUG DELIVERY SYSTEMS

(75) Inventors: Kamal RAMZIPOOR, Fremont, CA (US); Ary CHERNOMORSKY, Walnut Creek, CA (US)

Correspondence Address: LEVINE BAGADE HAN LLP, 2400 GENG ROAD, SUITE 120, PALO ALTO, CA, 94303, US

(73) Assignees: Laura N. Dietch, Menlo Park, CA (US); Ary Chernomorsky, Walnut Creek, CA (US); Kamal Ramzipoor, Fremont, CA (US)

(21) Appl. No.: 12/580,162

(22) Filed: Oct. 15, 2009

Related U.S. Application Data
(63) Continuation-in-part of application No. 11/852,711, filed on Sep. 10, 2007.
(60) Provisional application No. 61/105,749, filed on Oct. 15, 2008.
(60) Provisional application No. 60/868,915, filed on Dec. 6, 2006.

(51) Int. Cl. A61M 25/10

(52) U.S. Cl. 604/103.02

(57) ABSTRACT

Drug delivery systems which are configured to retain and then distribute one or more drugs upon an actuating surface and/or balloon for delivery to a tissue region are described herein. The drug delivery system may comprise in one variation a volume of one or more drugs held in a reservoir, e.g., a silo, which may be located proximally of the expandable actuating surface and/or balloon. The one or more drugs may be separated from one another by valves or immiscible fluid barriers for distribution upon the surface, which may be varied in pore distribution, have a coating or covering for facilitating drug distribution, etc.

### CROSS-REFERENCE TO RELATED APPLICATIONS

The application claims the benefit of priority to U.S. Prov. Pat. App. 61/105,749 filed Oct. 15, 2008, and is a continuation-in-part of U.S. patent application Ser. No. 11/852,711 filed Sep. 10, 2007, which claims priority to U.S. Prov. Pat. App. 60/868,915 filed Dec. 6, 2006, each of which is incorporated herein by reference in its entirety.

### FIELD OF THE INVENTION

The present invention relates to tissue expanding devices and methods that are removably placed upon a tissue region of interest in a human body to create an opening. The devices may have an actuating surface for delivery of various therapeutic agents into or upon the targeted site.

### BACKGROUND OF THE INVENTION

One of the most common techniques for treatment of vascular occlusive disease is called percutaneous balloon angioplasty or PTA. However, the PTA has a significant drawback that is the high potential for the stenotic vessel to re-close after the procedures, in 30% to 45% of the patients treated, a phenomenon known as re-stenosis. Hence, scaffolds called stents or stent grafts have been developed that stay in place to keep the vessel patent after dilatation. Despite this evolution, stenting is only able to decrease the re-stenosis rate down to 20% to 30% although with additional cost and clinical risks. Advances in drug eluding stents have significantly improved these outcomes by achieving further reduction of re-stenosis rates to the levels of 9%. Unfortunately, this has been eclipsed by reports of complications such as Late Stent Thrombosis, where the blood-clotting inside the stent can occur one or more year's post-stent implantation. While this has been seen rarely in currently marketed devices, thrombosis is extremely dangerous and potentially fatal in over 45% of the cases.

Late Stent Thrombosis usually occurs before endothelialization has been completed. For bare-metal stents, this process takes a few weeks. The drug-eluting stents inhibit re-stenosis by inhibiting fibroblast proliferation, but they also tend to delay the endothelialization process. Additionally the stents are covered with drug carrier polymers that themselves are often inflammatory to the tissue. Combinations of these two factors may cause a late or incomplete healing of the vessel wall leading to Late Stent Thrombosis.

A local drug delivery device which would deliver predetermined volume and concentration of drugs to the target while avoiding complications associated with the drug-eluting stents would be highly advantageous.

In fact there are several local drug delivery devices, including catheters with permeable balloon membranes and/or perfusion holes to aid with this delivery. However, most are plagued with the rather uniform problem of low transfer efficiency, rapid washout, poor retention, systemic toxicity and the potential for additional vessel injury.

In addition, many medical procedures require the surgical formation and maintenance of a cavity within a patient's body. For example, the treatment of certain tumors may require a multi-faceted approach that includes a combination of surgery, radiation therapy and chemotherapy. In such an approach, after an initial surgical procedure has been performed to remove as much of a tumor as possible, radiation and chemotherapy are performed to kill remaining cancerous cells that could not be removed surgically. These remaining cancerous cells are usually concentrated in an area surrounding the site of the surgery and can best be reached by inserting therapeutic materials directly into the surgery site, in close contact with the affected tissues.

In the case of radiation therapy, one of the more effective treatment methods is brachytherapy in which a source of radiation energy is placed within the body of the patient at the site of the removed tumor to substantially evenly treat the region that formerly surrounded the surgically removed tumor. In addition to or instead of radiation therapy, therapeutic chemical compounds may be used to kill cancerous cells located in the vicinity of a surgically removed tumor.

Therefore, it is generally desirable to be able to locally treat these types of cavities or lumens to effectively deliver any needed treatments.

Accordingly, there exists a need for methods and apparatus for effectively and efficiently delivering pharmaceutical agents to a specific location within the blood vessels or within body cavities or lumens (surgically created or otherwise) of a human body.

### SUMMARY OF THE INVENTION

Endovascular treatment of a stenotic lesion may be accomplished by a device that can expand the vessel via a balloon and deliver a therapy such as anti-restenotic and/or anti-thrombosis agents/drugs into the vessel wall. One variation may include a device that contains a balloon with a three-dimensional surface and significant capacity to deliver therapeutic agents/drugs into the vessel.

Such a device may also selectively deliver pharmaceutical agents at predetermined balloon diameters. Since the drug may be released at a given balloon diameter, infusion and washout during delivery and inflation periods may be eliminated, providing for a highly efficient and precise delivery mechanism. Moreover, often times it is desirable to have different agents to address different aspects of the stenotic lesion within the vessel, thus to the device may also be configured to provide for release of a first agent when the balloon reaches its first diameter and the second and third agents (or more), as necessary, when the balloon diameter increases. This is highly beneficial, for example, when encountering thrombosed and stenotic lesions where a device containing fibrolytic and anti restenotic agents can be used. Since presence of the thrombus causes reduction in vessel diameter, the fibrolytic agent may be first released when balloon researches its small diameter, dissolving the thrombus. The balloon may be then fully inflated, releasing the anti-restenotic agent into the vessel wall.

Another embodiment of the device is related to the release of different drugs or different concentrations of the same drug at a given balloon diameter. One example of the use of this feature is addressing edge effect restenosis. Current generation of drug eluting stents have problems with edge effect or restenosis beyond the edges of the stent and progressing around the stem into the interior luminal space.

The causes of edge effect restenosis in first generation drug delivery stents are currently not well understood. It may be that the region of tissue injury due to angioplasty and/or stent implantation extends beyond the diffusion range of current generation agents such as Paclitaxel or Rapamycin, which tend to partition strongly in tissue. Placing higher doses or higher concentrations of agents along the edges, placing different agents at the edges which diffuse more readily through the tissue, or placing different agents or combination of agents at the edges of the treated area may help to remedy the edge effect restenosis problem.

Another example of treatment may include treating a patients having thrombosed vessels, wherein the device is progressively expanded to various diameters, each time releasing a dose of fibrolytic agent dissolving thrombosis immediately surrounding the balloon until the entire lumen is cleared and a full recanalization is achieved.

Further examples of devices and methods which may be utilized herewith are described in further detail in U.S. patent application Ser. No. 11/852,711 filed Sep. 10, 2007 (U.S. Pat. Pub. 2008/0140002 A1), which is incorporated herein by reference in its entirety.

Yet another embodiment of the device is related to the release of different drug volumes, concentrations or different drugs from a reservoir, e.g., a silo or individual silos, that are located within the delivery system and are in fluid communication with the three dimensional surface of the balloon.

In a further variation, any of the treatment devices and/or methods described herein may be utilized for the treatment of body cavities or lumens, e.g., surgically created cavities such as those formed for treatment of cancer. One the embodiments in particular is further directed treating tissue surrounding a surgically created resection cavity after surgical treatment of, e.g., malignant breast cancer. Generally, one of the described devices having a distal end of a catheter equipped with a modified 3D dynamic surface containing, e.g., non-conductive elements, conductive mesh, silo reservoir with a anticancer therapeutic agent, etc., may be inserted into the resection cavity to deploy an inflatable element at a desired location within the resection cavity in such a manner that, in this variation, non-conductive elements of the balloon surface may come into contact with inner surfaces of the resection cavity for treatment.

### BRIEF DESCRIPTION OF THE DRAWINGS

FIG. 1A shows an illustrative view of vessel of a patient body with a variation of the treatment system minimum invasively positioned therein.

FIG. 1B shows a partial cross-sectional detail view of a variation of the catheter apparatus having a balloon with a surface for expanding and temporarily contacting and delivering pharmaceutical agents/drugs into the vessel wall.

FIG. 2A shows a partial cross-sectional view of the catheter apparatus placed within a vessel

FIG. 2B shows a partial cross-sectional view of the catheter with a balloon having a surface and at least partially expanded within a vessel.

FIG. 2C shows a partial cross-sectional view of the balloon having an absorbent surface and fully expanded and apposed against the interior of the vessel releasing agents/drugs into the vessel wall.

FIG. 3 illustrates release of different agents or different concentrations of the same agent at locations distal and proximal to the balloon to address such disorders as “edge effect restenosis”.

FIG. 4A illustrates a catheter apparatus with a balloon having a surface with longitudinal segments capable of un-compressing when the balloon diameter is relatively small.

FIG. 4B illustrates a catheter apparatus with a balloon partially expanded, releasing fibrolytic agents.

FIG. 4C illustrates a catheter apparatus with a balloon fully expanded, releasing anti-restenotic agents.

FIGS. 5A to 5C show a cross-section view of the drug delivery balloon with outer porous layer going through the inflation process with the consequent changes in the pore architecture and dimensions.

FIG. 5D shows an enlarged cross sectional view of the balloon segment having an outer layer containing predetermined pore architecture.

FIGS. 6A and 6B show longitudinal views of the enlarged segments of a porous layer having another predetermined pore architecture.

FIGS. 6C and 6D show enlarged segments of a porous layer that includes a plurality of porous fibers having yet another predetermined pore architecture.

FIG. 7A illustrates an example of the stacked structure of a porous layer.

FIG. 7B illustrates another example of yet another stacked structure with different pore architecture and orientation of a porous layer.

FIG. 8A is a photomicrograph of a porous layer having a predetermined pore architecture.

FIG. 8B is another photomicrograph of a porous layer having a predetermined pore architecture.

FIG. 8C is another photomicrograph of a porous layer having a predetermined pore architecture.

FIG. 8D is yet another photomicrograph of a porous layer having another predetermined pore architecture.

FIG. 8E is another photomicrograph of having still another predetermined pore architecture.

FIG. 9A is a combination of photomicrographs of porous layers illustrating the formation of a stacked laminate structure including a first layer having a first predetermined pore architecture and a second layer having a second predetermined pore structure.

FIG. 9B is a combination of photomicrographs of a porous layer that collectively illustrate a predetermined pore density gradient and/or predetermined size gradient.

FIGS. 10A to 10C illustrate delivery and release of a stent in combination with infusion of a therapeutic agent into the targeted site.

FIG. 11A is a perspective view of the three-dimensional substrate sleeve.

FIG. 11B is a perspective view of the substrate sleeve placed on the catheter balloon which shows the three-dimensional porous nature of the substrate.

FIG. 11C is a longitudinal view of the substrate sleeve fitted on the balloon

FIG. 11D is an enlarged longitudinal view of the substrate sleeve fitted on the balloon in its inflated state and shows the configuration of the pores throughout the thickness of the substrate wall.

FIG. 12A shows the substrate sleeve covered with a polymeric film.

FIG. 12B illustrates the expansion of the balloon and as a consequence of that disintegration and defragmentation of the coating film turning it into a disintegrated surface.

FIG. 12C shows further disintegration of the coating film into even smaller fragment which are either soluble or degradable by the physiological environment.

FIG. 12D show a fully inflated balloon covered with a substrate sleeve completely free of coating.

FIGS. 13A to 13C illustrate additional variations of the expandable balloon covered with a sleeve which have various configurations for reservoirs along the sleeve surface which are capable of expanding when the balloon reaches a predetermined diameter to release any biologically active substances.

FIG. 13D illustrates a cross-sectional end view of the balloon having an outer layer and an example of reservoir architecture.

FIGS. 14A and 14B show perspective and cross-sectional end views, respectively, of another variation for reservoir configuration.

FIG. 15 is a graph showing an increase in pore size and correlated release of a drug agent when the balloon reaches its maximum diameter.

FIG. 16 is a graph showing the maximum release of a drug agent at a predetermined balloon diameter of, e.g., 4 mm.

FIG. 17 is a graph showing an example of two different pore architectures responding to the balloon expansion.

FIG. 18 is a graph showing 100% release of a first drug agent when balloon researches its first diameter of, e.g., 3 mm, followed by complete release of a second drug agent when the balloon is fully inflated to, e.g., 4 mm diameter.

FIGS. 19A to 19D show cross-sectional views of the drug delivery balloon with outer porous layer covered with outer sheath with structurally jeopardized surface, going through the inflation process with the consequent changes in the pore architecture and dimensions and the outer sheath that disintegrates under radial stresses generated during inflation of the balloon.

FIGS. 20A and 20B illustrate an outer sheath with structurally jeopardized surface were longitudinal cut are pre made to accelerate a peel-off process.

FIGS. 21A and 21B illustrate an outer sheath with a structurally jeopardized surface having multiple perforations or holes to allow elution of the biological agent under pressure.

FIGS. 22A to 22D illustrate an outer sleeve made from a thin layer of biodegradable material with a mechanically jeopardized surface having multiple cuts and/or holes to accelerate the process of bioabsorption under pressure to allow elution of the biological agent.

FIGS. 23A to 23D illustrate an outer sleeve made out of a thin layer of material which is degradable under application of energy.

FIG. 24A shows an illustrative view of vessel of a patient body with a variation of the treatment system minimum invasively positioned therein.

FIG. 24B shows a partial cross-sectional detail view of a variation of the catheter apparatus having a balloon with a surface for expanding and temporarily contacting and delivering pharmaceutical agents/drugs into the vessel wall.

FIGS. 25A to 25D show side views of a variation of a drug delivery system where a volume of one or more drugs held in a reservoir, e.g., a silo, may be located proximally of an expandable actuating surface and/or balloon.

FIGS. 26A to 26C show side views of another variation where the one or more drugs may be contained in multiple silos which are separated from one another.

FIGS. 27A to 27C show side views of another variation where a gradient in the pore distribution may be varied over the surface to distribute the one or more drugs.

FIGS. 28A to 28C show side views of yet another variation where a coating or covering may be placed over at least a portion of the surface and/or balloon, e.g., mid and/or proximal portions, to facilitate distribution of the one or more drugs over the surface.

FIG. 29A depicts a drug eluting delivery system comprising internal drug containing lumen, partially filled with therapeutic agent, inflatable balloon with “3D smart surface” (in deflated state), fluid for plunger operation, radio opaque plunger, and radio opaque graduation markers.

FIG. 29B depicts a drug eluting delivery system in action where “3D smart surface” activated by inflation balloon into working state, then pushing fluid and moving plunger.

FIG. 30A depicts a drug eluting delivery system comprising internal lumen, partially filled with different therapeutic agents, inflatable balloon with “3D smart surface” (in deflated state), fluid for plunger operation, radio opaque plunger, and radio opaque graduation markers.

FIG. 30B depicts a drug eluting delivery system in action where “3D smart surface” activated by inflation balloon into working state, then pushing fluid and moving plunger.

FIG. 30C depicts a process of banding of the collapsible solid separator under hydraulic pressure exerted by the plunger.

FIGS. 31A to 31D depicts an embodiment of the drug eluting delivery system comprising “3D surface”, silo drug reservoir and conductive mesh.

FIGS. 32A to 32D depicts an embodiment of the drug eluting delivery system comprising “3D surface” and conductive mesh.

FIG. 33A depicts a composition of the positively charged particle comprising Paclitaxel, dispersed in PGA-PLA copolymer, encapsulated in the Chitosan matrix.

FIG. 33B depicts a composition of the negatively charged particle comprising Paclitaxel, coupled with Albumin.

FIG. 34A and FIG. 34B show cross-sectional schematic diagrams illustrating a modified 3D dynamic surface having an anticancer therapeutic agent stored in a silo reservoir of a delivery system.

FIGS. 35A and 35B show cross-sectional illustrations showing an example of delivery, placement, and subsequent expansion of a treatment balloon in a surgically formed cavity in, e.g., a breast, using a trocar introducer.

FIG. 36 schematically shows an example of an expanded treatment balloon having a plurality of non-conductive elements which is expanded for treatment within a surgically formed cavity.

FIG. 37 schematically shows another example of an expanded treatment balloon having non-conductive elements on the surface of the balloon positioned within a cavity such as a uterus.

### DETAILED DESCRIPTION OF THE INVENTION

Although devices and methods are described relative to a biologically active substance applied to the interior of the blood vessel device, it is to be understood that the other variations are not to be limited thereby. Indeed, other variations may be advantageously utilized for simultaneous angioplasty and anti-restenosis treatment of various blood vessels.

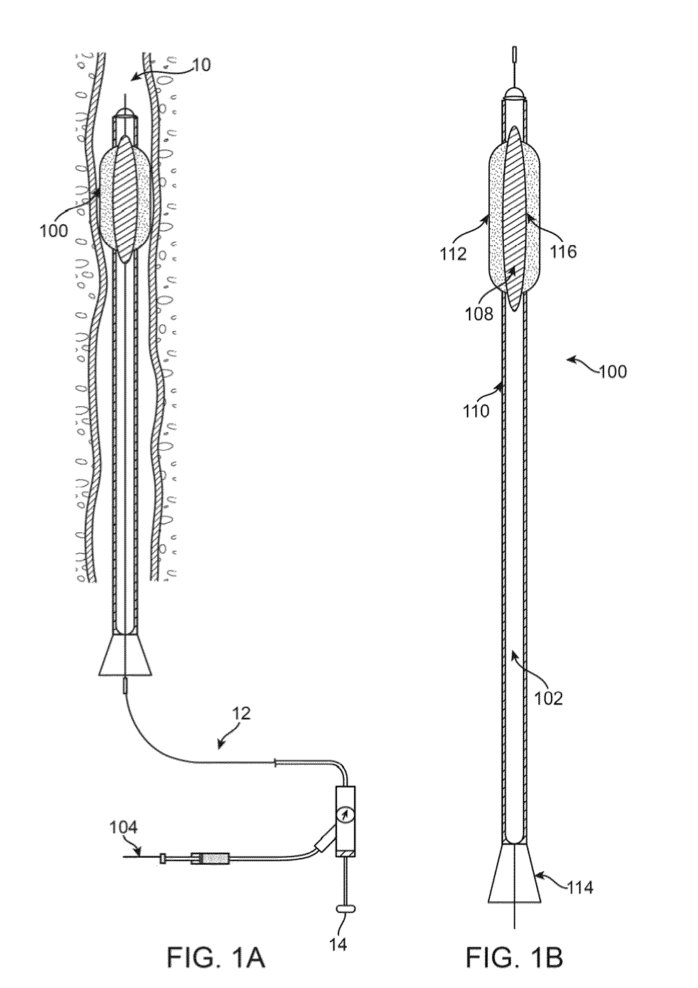

FIG. 1A illustrates an illustrative view of a blood vessel 10 of a patient body with one variation of the catheter treatment system 100 positioned therein. The catheter treatment system 100 may be introduced into the patient body via percutaneous access through the patient's skin and into the blood vessel 10 to be treated. The catheter system 100 may be advanced into the blood vessel 10 until the portion to be treated has been reached and/or traversed by the catheter system 100. As further shown, the catheter treatment system 100 may be connected via an inflation/deflation tubular member 12 to a pump 14 positioned externally of the patient.

Moreover, one or more access ports may be incorporated with the system to allow for access by other devices, such as guidewire 104, which may be optionally advanced distally of the catheter system 100 to facilitate access through the blood vessel. Additionally, a proximal portion 114 of the catheter assembly 100 may further define a flared or tapered portion to facilitate the insertion and access of a guidewire 104 into and through the assembly 100.

FIG. 1B illustrates one variation of an elongated tubular catheter assembly 100, having a distal and a proximal end and a lumen 102 to optionally receive a guidewire 104 therethrough. The catheter assembly 100 also includes an inflation balloon 108, and an inflation lumen 110 that is in fluid communication with the balloon 108. The outer surface 116 of the balloon 108 may be completely or at least partially covered with a highly absorbent material such as foam 112 or other absorbent materials, as further described below. The outer surface 116 of the balloon 108 may be comprised of a retaining material to facilitate the absorption and retention of an agent/drug therein. Such a retaining material may include any number of substances which are configured to retain and/or absorb a biological or non-biological liquid or solid medium. Such materials may be accordingly configured to include a number of reservoirs for retaining the liquid or solid medium where reservoirs may include any liquid or solid medium retaining structures, e.g., pores, troughs, capacitors/capacitance (which used herein may refer to the ability of a liquid or solid medium retaining structure to hold or store that medium).

The retaining material is designed to react to the force applied by expansion of the balloon 108. When the balloon is in deflated state, the pores are closed under the compression that naturally exists within the property of the material, effectively retaining the agent/drug therein. However the force with which the expanded condition of the balloon exerts radially, will un-compress the pores, releasing therapeutic agents to the site. In many instances, varying such material characteristics, including but not limited to: tensile strength, stiffness, Young's Modulus, etc., may vary the force applied by the balloon expansion. One skilled in the art can design a retaining material with particular desired characteristics to un-compress by the force that is applied when balloon reaches a specific diameter. For example, when treating a 3 mm vessel diameter, the porous surface un-compresses only when the balloon expands to that specific diameter, thereby preventing premature infusion, diffusion and maintaining the original drug load during delivery and inflation of the device.

Further examples of devices and methods which may be utilized and integrated with the systems described herein are shown and described in further detail in U.S. patent application Ser. No. 11/461,764 filed Aug. 1, 2006, which is incorporated herein by reference in its entirety.

Once the catheter system 100 has been advanced and desirably positioned within the vessel to be treated, the agents/drugs contained within the outer retaining surface 112 may be applied to or against the interior of the vessel to be treated, as further described below.

Although a single balloon 108 is illustrated, one or more balloons positioned in series relative to one another may alternatively be utilized. Each of the balloons may be connected via a common inflation and/or deflation lumen to expand each of the expandable members. Alternatively, each of the balloons may be connected via its own inflation/deflation lumen such that individual balloons may be optionally inflated or deflated to treat various regions of the vessel.

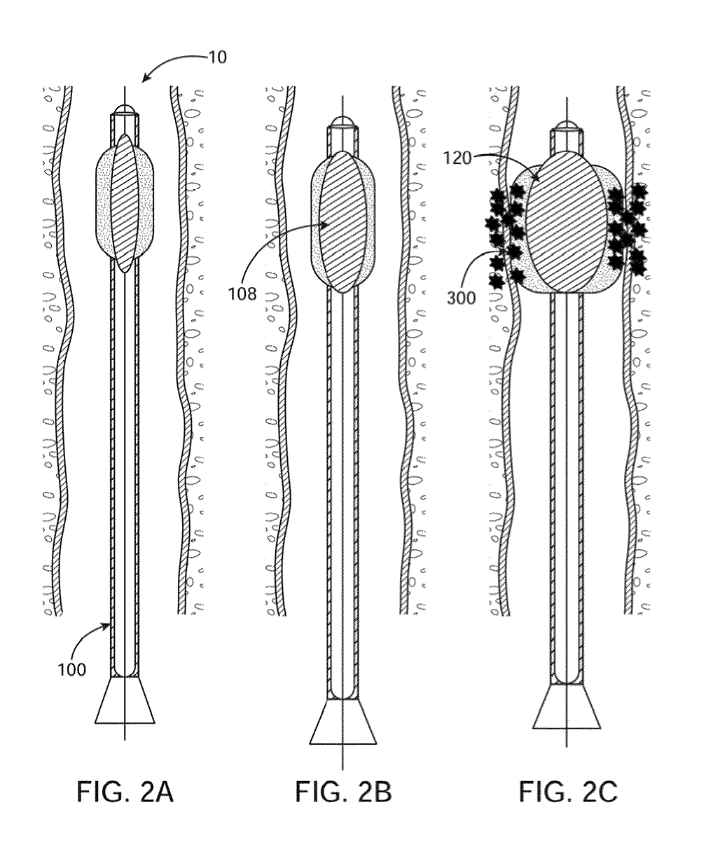

FIG. 2A shows the catheter assembly 100 introduced into the vessel and advanced to the location to be treated. Once desirably positioned adjacent to or proximate to the vessel 10 to be treated, the balloon 108 may be inflated via pump 14 through inflation/deflation tube 12, as shown in FIG. 2B, and appose its porous surface 112 uniformly or otherwise against the interior wall of the vessel 10. Pressure from the balloon 108 will un-compress pores of the surface 120, causing release of the agents 300, directly, uniformly (or non-uniformly), and efficiently to the vessel with minimum dilution and diffusion, shown in FIG. 2C.

Once the desired agents/drugs have been applied for a desired period of time, the catheter system 100 may be deflated and removed from the vessel.

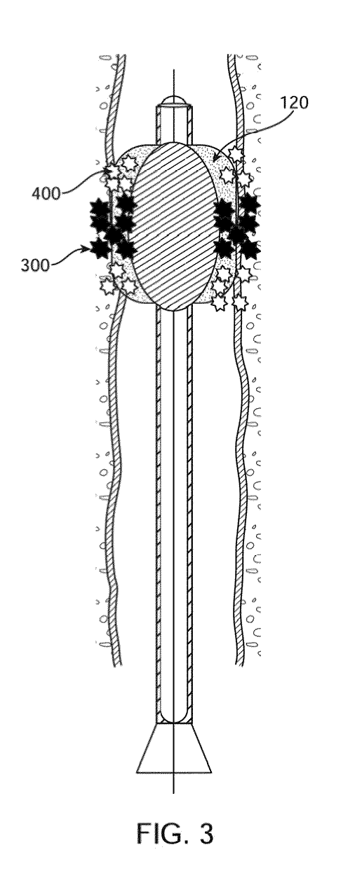

FIG. 3 show another variation of the retaining surface 112, having one drug agent 300 at its center and different agents or different concentration of the same agent/drug 400 at its proximal and distal ends to address “edge effect re-stenosis”.

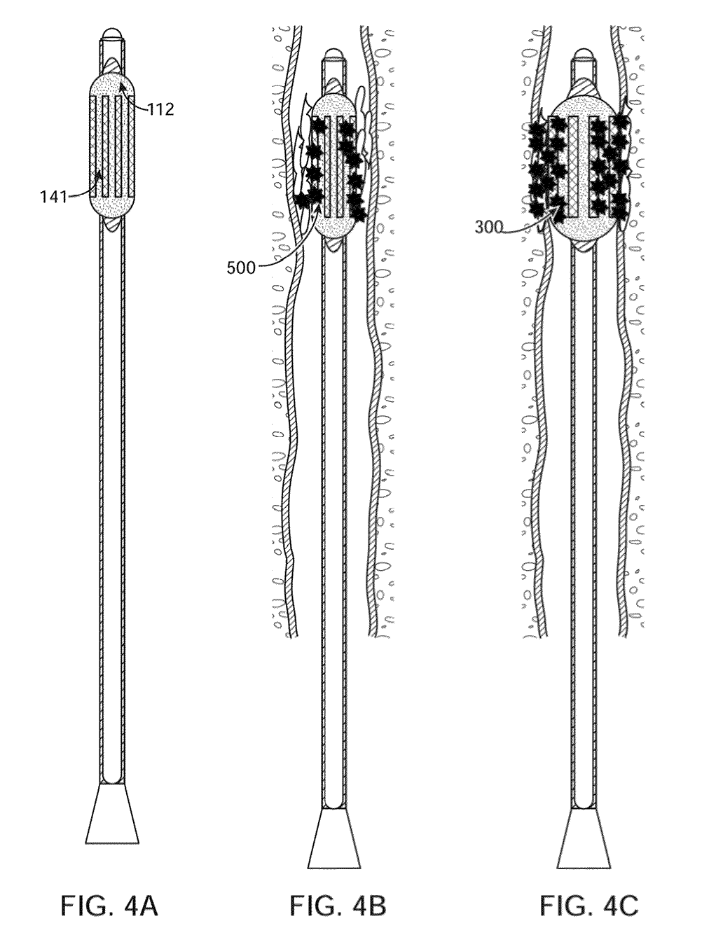

FIG. 4A illustrates yet another variation of the retaining surface 112, capable of releasing different agents or different concentration of the same agent at different balloon diameters. This is accomplished by the porous surface 112 having longitudinal sections 141 capable of un-compressing when balloon is inflated to its first diameter, thereby releasing the first drug that in present example is a fibrolytic agent 500 to dissolve the thrombus within the stenotic lesion of the vessel as shown in FIG. 4B. Further inflation of the balloon will un-compress the remaining segments of the porous surface that contain anti-restenotic agent 300 and release such agents to the vessel wall, shown in FIG. 4C.

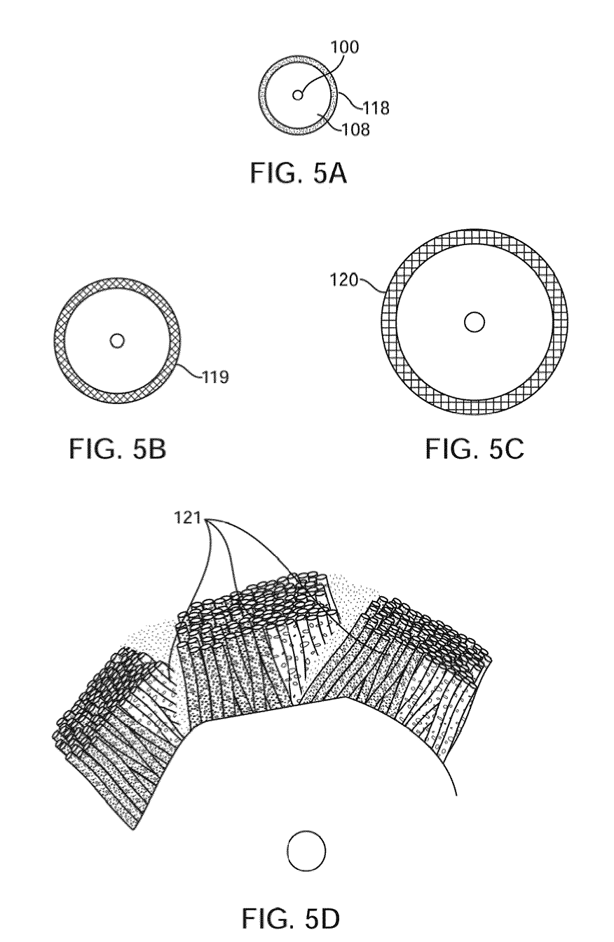

As shown in FIG. 5A, the treatment device 100 is a balloon 108 coupled with an outer porous layer 118. The treatment device 100 is positioned such that the balloon 108 coupled with outer porous layer 118 is adjacent to the target lesion. The assembly may then be inflated and expanded as shown in FIGS. 5B and 5C by infusing an inflatable agent such as saline. As the assembly is inflated and expanded, the outer porous layer 119 is stretched. As shown at FIG. 5C, the initial pore configuration may then be changed while the balloon remains inflated, causing the biological substance entrapped in the cell of the stretched or otherwise deformed porous layer 120 to become available for the contact with a targeted tissue.

Further variations may include a microporous cross-linked polymer matrix having a predetermined pore architecture. A “pore” may include a localized volume of the outer layer that is free of the material from which the outer layer is formed. Pores may define a closed and bounded volume free of the material from which outer layer is formed. Alternatively, pores may not be bounded and many pores may communicate with one another throughout the internal matrix of the present outer layer. The pore architecture, therefore, may include closed and bounded voids as well as unbounded and interconnecting pores and channels. The internal structure of the outer layer defines pores whose dimensions, shape, orientation and density (and ranges and distributions thereof), among other possible characteristics are tailored so as to maximize the capacity of the treatment device to contain and deliver under pressure certain biological substances. There are numerous methods and technologies available for the formation matrices of different pore architectures and porosities. By tailoring the dimensions, shape, orientation and density of the pores of the outer layer, a capacity to absorb and release biological agents in certain predictable manner may be formed that may be used for local drug delivery.

An embodiment of the outer layer may be formed of or include a polyurethane matrix having a predetermined pore architecture. For example, the outer layer of the treatment device may include one or more sponges of porous polyurethane having a predetermined pore architecture. Suitable polyurethane material for the outer layer of the treatment device may be available from, for example, Lendell Manufacturing, Inc.; Hi-Tech Products (Buena Park, Calif.), PAC Foam Products Corp. (Costa Mesa, Calif.), among others. Moreover, the outer layer may be comprised of any number of suitable materials including, but not limited to, elastomeric and non-elastomeric polymers such as polyurethane, silicone, pebax, polyimide, polyethylene, polyetheretherketone (PEEK), polyvinylidene fluoride (PVDF) liquid crystal polymer (LCP), family of fluoropolymers such as polytetrafluoroethylene (PTFE), expanded polytetrafluoroethylene (ePTFE), family of polyesters such as Hytrel, Polyethylene terephthalate (PET), polybutylene terephthalate (PBT) and their copolymers, etc. The outer layer of the treatment device may, according to further embodiments, be used to medically treat the patient. That is, the porous matrix of the outer layer may be imbibed or loaded with a therapeutic agent to deliver the agent through elution at the interior of the vessel wall. Such a therapeutic agent may include, for example, biopharmaceuticals, therapeutic agents or physiological process modifying agents which can be anti-infective, anti-inflammatory, anti-proliferative, anti-angiogenic, anti-neoplastic, anti-scarring, scar-inducing, tissue-regenerative, anesthetic, analgesic, immuno-modulating agents and neuro-modulating, bioadhesives, tissue sealants and sclerosing agents, to name but a few of the possibilities.

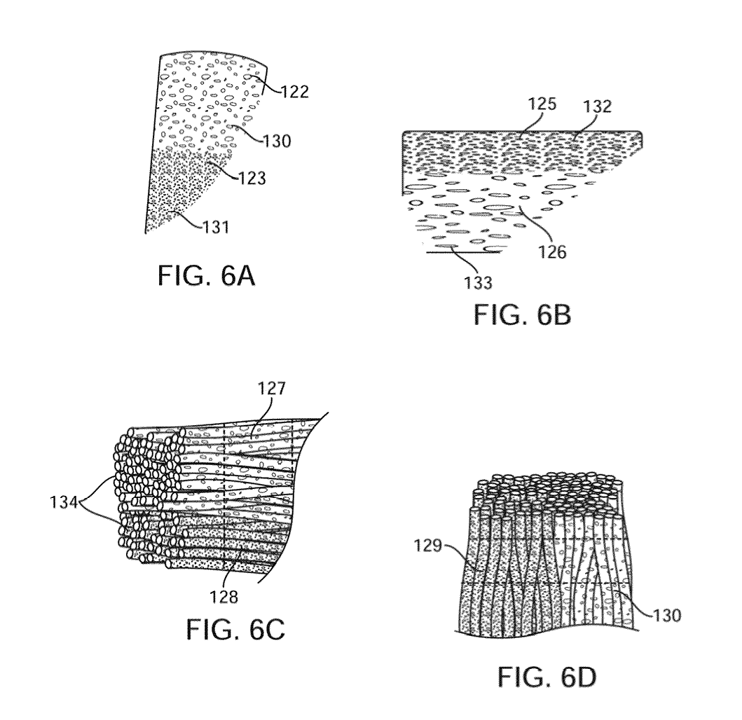

The outer layer 121 shown in FIG. 5D may be formed of one or more thin sheets or fibers of polyurethane or silicone material having a predetermined (and controlled) pore architecture that has been coupled with the outer surface of the balloon. FIG. 6A shows an outer layer 121 having predetermined pore architectures. As shown therein, the outer layer 121 may include a first portion 122 and a second portion 123. The polymer matrix of the first portion 122 of the outer layer 121 defines a plurality of pores 130 having a first predetermined pore architecture and the polymer matrix of the second portion 123 of the outer layer 121 defines a plurality of pores 131 having a second predetermined pore architecture. The dimensions of the layers or portions may be selected at will, preferably accounting for the dimensions of the treatment device. As shown, the first pore architecture features pores 131 that are relatively small, have a narrow pore size distribution and are substantially randomly oriented. In contrast, the second pore architecture features pores 130 that have a relatively larger size, have a wider pore size distribution, and are less densely distributed than the pores 130 of the first portion 122 of the outer layer of the treatment device.

FIG. 6B shows a segment of outer layer 121 having an alternative predetermined pore architecture. As shown, the outer layer includes a first portion 125 and a second portion 126, each of which has a predetermined pore architecture (pore 132 in first portion 125 and pore 133 in second portion 126, which in this example illustrates pores 132 having a smaller size relative to pores 133). It is to be noted that the present outer layer may have more than the two portions. The first portion 125 is stacked on the second portion 126. As with the embodiment shown in FIG. 6A, the first and second portions may have pore architectures that facilitate optimal drug absorption characteristics. The different pore architectures of the outer layer may also be chosen so as to maximize the controlled drug release when the balloon 108 is fully expended and positioned against the targeted lesion.

FIGS. 6C and 6D show various other configurations for the porous outer layer. As shown therein, embodiments may include or be formed of a bundle of fibers or fibrils 134 of (for example) polyurethane material having one or more predetermined pore architectures. The pores 127, 128, 129 defined within the polyurethane matrix of all or some of the fibers are shown in the various figures herein.

As shown in FIG. 6C, two or more bundles of fibers of polyurethane material (for example, the fibers may be made of or include other materials) may be used in the formation of outer layer. As shown, the pores within the fibers of the first bundle 127 may collectively define a first pore architecture, whereas the pores within the fibers of a second bundle 128 may collectively define a second pore architecture that is different from the first pore architecture. The two bundles may then be joined together, for example, by re-wetting the bundles, stacking them and lyophilizing the composite structure. The length and diameter of the fibers may be selected and varied at will. The fibers or bundles thereof may even be woven together. From this composite structure, outer layer may be formed. As shown in FIGS. 6C and 6D, the bundles of fibers may be arranged and oriented in a different manner for example perpendicular or parallel to the surface of the balloon.

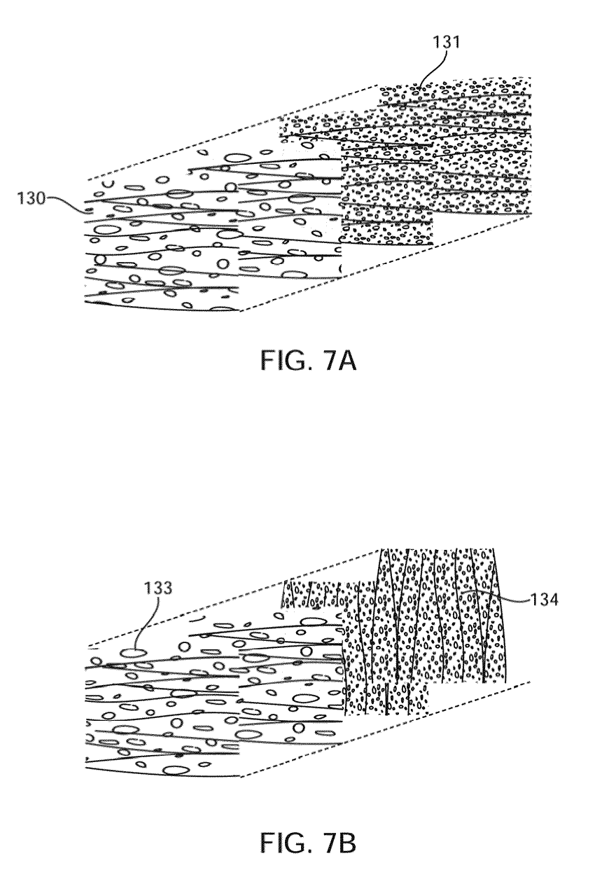

As shown in the exploded views of FIGS. 7A and 7B, the outer layer may have a layered laminate structure in which sheets formed of fibers (or woven fibers) having a first pore architecture are stacked onto sheets formed of fibers having a second pore architecture. As shown in FIG. 7A, many variations on this theme are possible. As shown therein, the orientation of the fibers (and thus of the pores defined by the polymer matrix thereof) may be varied. For instance, whereas the fibers of the first (top or outer, for example) portion of the outer layer may be oriented in a first direction, whereas the fibers of the second (bottom or inner, for example) portion of the outer layer may be oriented along a direction that is different from the first direction (perpendicular thereto, for example).

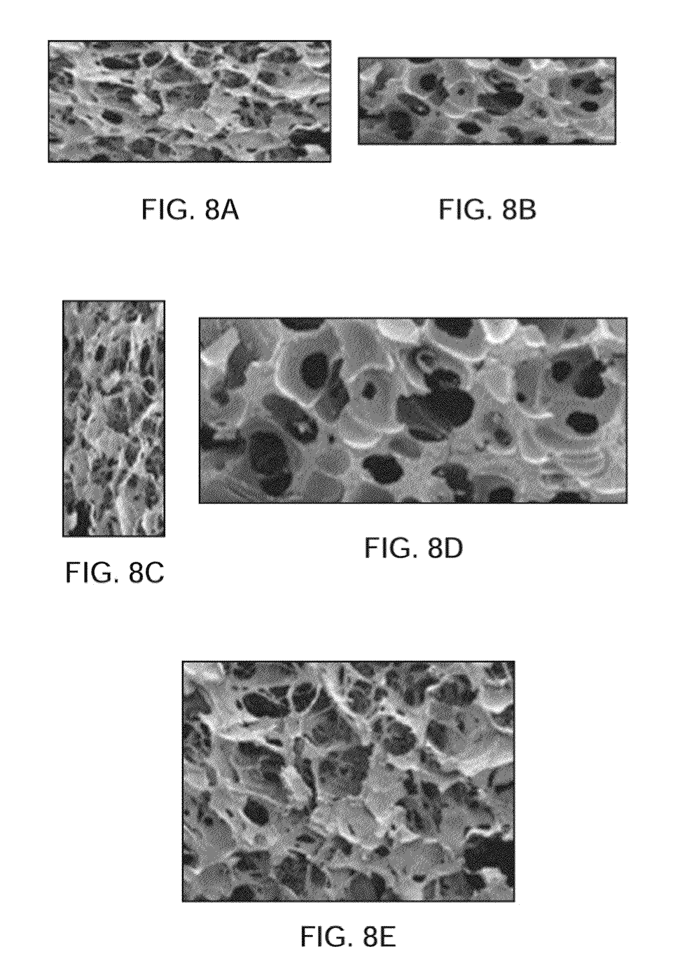

FIGS. 8A to 8E are photomicrographs of polymeric matrices having various pore architectures that can be generated using various technologies such as lyophilization or usage of a foaming agents, just to mention a few.

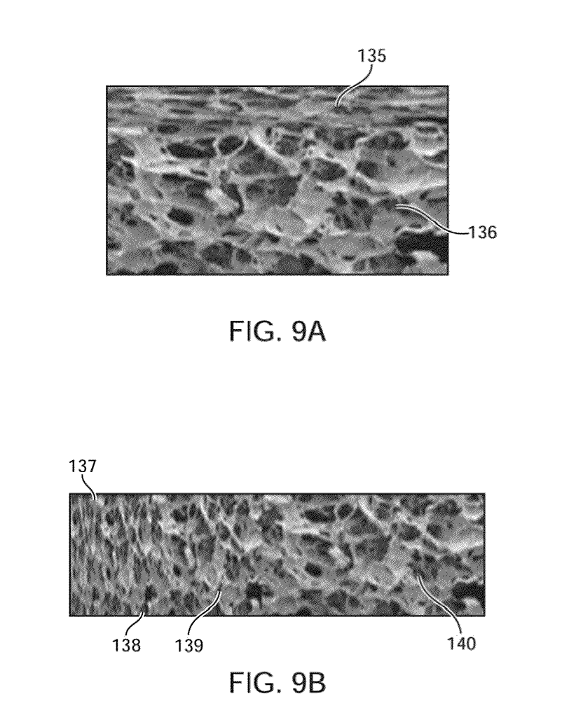

FIGS. 9A and 9B are combinations of photomicrographs to illustrate further embodiments of the outer layer. FIG. 9A shows an outer layer 121 that includes a first portion 135 having a first pore architecture and, stacked thereon, a second portion 136 having a second pore architecture. As shown; the pore architecture of the first portion 135 may be characterized as being relatively denser than the pore architecture of the second portion 136. Alternatively, the outer layer 121 may be structured such that the first portion has a higher porosity (is less dense) than that of the second portion 136. The thicknesses of the first and second portions 135, 136 may be varied at desired. More than two layers of polymeric material may also be provided.

FIG. 9B shows an outer layer 121 having a graduated porosity profile. Such an outer layer may be formed by lining up a plurality of polymer matrices having of progressively lower densities. That is, matrix 137 has the highest density (amount of polymer per unit volume), matrix 138 has the next highest density, matrix 139 has the next to lowest porosity and matrix 140 has the lowest porosity of the entire outer layer.

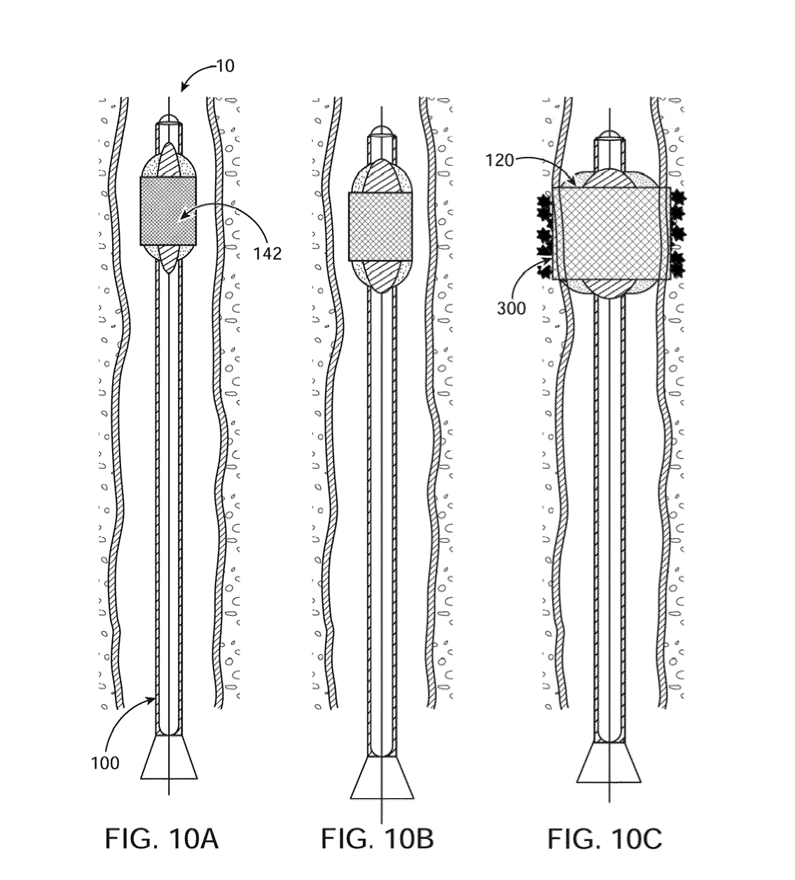

FIGS. 10A to 10C show the treatment device 100, delivering a stent 142 and simultaneously infusing a therapeutic agent into the targeted site.

A three-dimensional internal geometry and capability for retention or release of its contents is desirable. Such retention or release of substances are dependent on the type of application and the amount of the hoop stress required for the substrates in order to provide an effective local drug delivery of a prescribed dose to a targeted tissue. The substrate can be built or coupled to the surface of the balloon or produced in the form of a sleeve that can be fitted upon the balloon. Such porous substrate sleeves can be processed by several techniques well known in the fields of polymer processing and tissue engineering.

One of the methodologies of formation of porous polymer structures involves the mixing of water soluble inorganic salts into polymer-solvent systems and forming a tubular structure of a desired but limited thickness by one of many procedures available. The resulting polymer network is then cured and leached of salt by soaking in an aqueous solution.

Yet another method for forming a porous polymer substrate sleeve involves freezing water dispersion of a polymer at a certain regime so that water crystals of a certain size and shape are formed. The resulting frozen polymer network is then freeze-dried and water crystals are sublimated by application of a vacuum.

Also, foaming agents such as cyclopentane and blowing agents such as certain chlorofluorocarbons (CFCs), just to mention a few, can be used to produce “pseudo-porous structures”, i.e., to produce a closed pore cellular structure to the polymeric substrate sleeve.

Yet another method for forming a porous polymer substrate sleeve is utilization of mandrel dipping. Mandrel dipping methods can result in substrates which are limited to simple, thin-walled porous substrate material. Reproducibility and uniformity of the porous structures formed by dipping is typically tightly controlled.

Yet another method for forming a porous polymer substrate can utilize certain techniques similar to those employed for a formation of a porous graft particularly adapted for cardiovascular use, as described in U.S. Pat. No. 4,759,757 entitled “Cardiovascular graft and method of forming same”, which is incorporated herein by reference in its entirety. The described method generally comprises choosing a suitable, non-solvent, two component, hydrophobic biocompatible polymer system from which the graft may be formed; choosing suitable water soluble inorganic salt crystals to be compounded with the biocompatible polymer system; grinding the salt crystals and passing same through a sieve having a predetermined mesh size; drying the salt crystals; compounding the salt crystals with the biocompatible polymer system; forming a tube from said compounded salt and polymer system by reaction injection or cast molding; and leaching the salt crystals from the formed tube with water, said leaching of said salt crystals providing a tube with a network of interconnecting cells formed in the area from which the salt crystals have been leached.

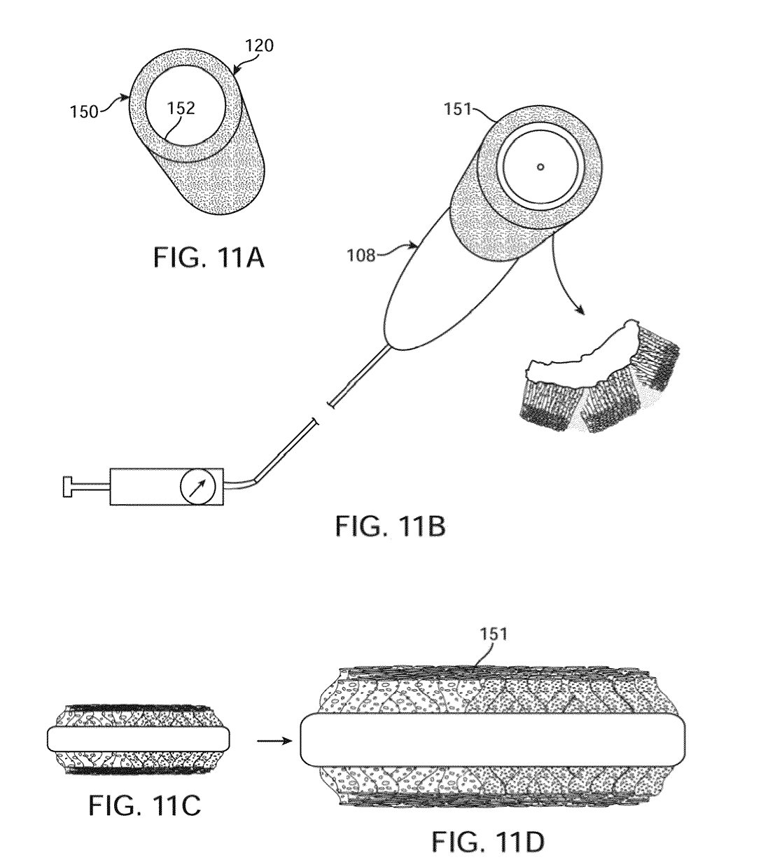

All of the above methods are suitable for the three-dimensional substrates manufacturing. Now referring to the drawings in greater detail, a sleeve 150 is illustrated in FIG. 11A which has a tubular configuration within an inner surface 152 and an outer surface 151 and is formed of a porous biocompatible polymer material with the surface 152 and 151 having cells or pores 120 therein. Referring now to FIG. 11B, there is illustrated a perspective view of the substrate sleeve 150 introduced upon the balloon 108 and a side view in FIG. 11C.

Referring now to FIG. 11D, there is illustrated therein an enlarged longitudinal view of the substrate sleeve fitted on the balloon. In this view is illustrated the honeycomb arrangement of the cells or pores 120. In this respect, by forming the sleeve 150, the cells or pores 120 within the sleeve are formed so that they interconnect throughout the wall thickness to form a porous network through the wall to the sleeve 150. This honeycomb network arrangement in a porous biocompatible polymer facilitates elution of a loaded biological substance into a substrate upon applying a certain hoop stress by the inflated balloon 108.

Referring now one of the suggested method for forming the substrate sleeve 150, it is first to be noted that the biocompatible polymer system from which the substrate sleeve is manufactured is a two component polymer system including polymers such as polyurethane, silicone and polytetrafluorethylene and a curing agent. Also, other hydrophobic polymer systems may be utilized and the choice of materials should not be confined to these three polymers. In such a two component polymer system, the first component is a resin, such as a silicone resin, and the second component is a curing agent/catalyst such as, for example, platinum. Other curing agents/catalysts available for use in such two component systems are tempered steel, heat, crosslinkers, gamma radiation, and ureaformaldehyde. As described above, it will be noted that this two component system is a non-solvent system. That is, the two components react together in the presence of salt, which is compounded with the two component system as described below. The two components are not a polymer and a solvent.

Once an appropriate two component polymer system has been chosen, it is compounded with a water soluble inorganic salt such as, but not confined to, sodium chloride. The size and shape of the pores 120 of the honeycomb network are dictated by the choice of the specific inorganic salt that is compounded with the polymer system. Typically, the crystals of salt chosen are ground and then put through a sieve whose chosen mesh size corresponds to the size requirement for the pore diameter to be utilized in the graft 10. The salt crystals are then placed in a drying oven at 135° C. for a period of, e.g., no less than 24 hours. The polymer system is then processed according to the method recommended by the manufacturer of the particular polymer system utilized and the dried salt crystals are mixed with the polymer system and compounded. The porosity and flexibility of the substrate sleeve 150 is dependent upon the ratio of water soluble inorganic salt to the polymer system with this ratio ranging anywhere from 25-755 by weight.

Once compounded, the water soluble inorganic salt and polymer are injection molded or reaction injection molded to form a tube of known inner and outer diameter. If desired, the tube can be extruded. Once the salt filled polymer tubes are formed, they are leached in water, dissolving the salt crystals and leaving a porous network of interconnecting cells 151, as illustrated in FIG. 11D. This method of formation provides for the rapid and reproducible formation of simple geometries within thin walled substrate sleeves as well as large, intricate geometries within thick walled substrate sleeves as dictated by the size of the anatomical structures in which the substrate sleeves is to be utilized.

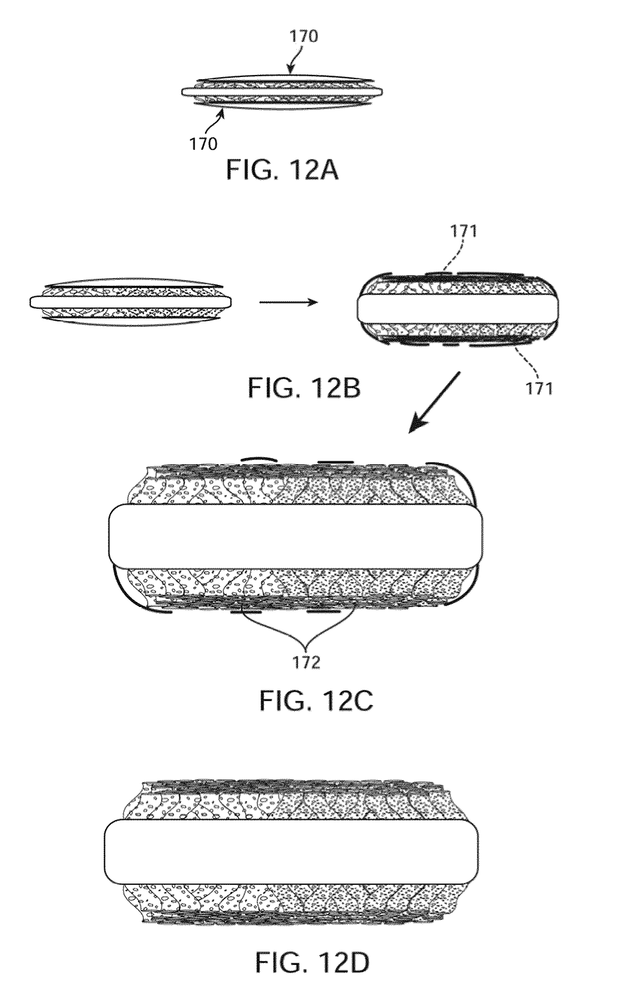

FIGS. 12A to 12D illustrate yet another embodiment where a thin layer of a polymeric biodegradable film 170 is placed on the outer surface of the substrate sleeve thereby preventing any undesirable leakage of the biologically active substance coupled with the substrate. FIG. 12B illustrates the expansion of the balloon and as a consequence of that disintegration and defragmentation of the coating film 170 turning it into a disintegrated surface 171. Biodegradable coating 170 can be formed with a variety of the biopolymers such as, but not limited to, synthetic and naturally occurring polymers including hydrophilic and hydrophobic synthetic polymers, small molecular weight crosslinkers having at least two carbon atoms, proteins, polysaccharides, lipids, DNA and their derivatives. Hydrophilic polymers may include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propyene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; polyacrylamides) such as polyacrylamide per se, poly(methacrylamide), poly(dimenthylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazonines, including poly(methyloxazoline) and poly(ethyloxazoline); and polyvinylamines. Naturally occurring hydrophilic polymers may include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans; e.g., hyaluronic acid; chitin chondroitin sulfate A, B, or C, keratin sulfate, keratosulfate and heparin; and activated polysaccharides such as dextran and starch derivatives, etc.

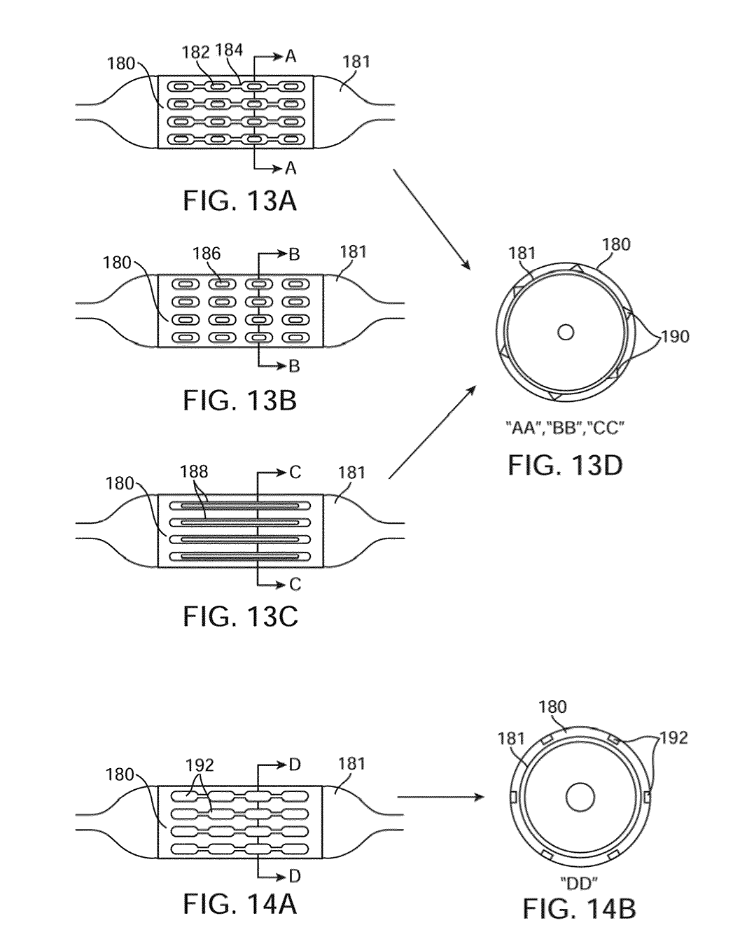

FIGS. 13A to 13C illustrate additional variations of the expandable sleeve 180 placed upon an expandable balloon 181 and which have various configurations for reservoirs along the sleeve surface which are capable of expanding when the balloon 181 reaches a predetermined diameter to release any biologically active substances. One example is illustrated in the perspective view of FIG. 13A where a plurality of individual reservoirs 182 interconnected via channels 184 may form a network of reservoirs over the sleeve surface. The individual reservoirs 182 may be uniformly spaced over the sleeve surface or scattered in various patterns depending upon the desired release results. Another variation is shown in FIG. 13B which illustrates a plurality of independent reservoirs 186 spaced over the sleeve surface uncoupled from one another. Yet another variation is illustrated in FIG. 13C which illustrates a variation where reservoirs 188 are configured to extend longitudinally along the surface of sleeve 180. Although the reservoirs are illustrated as being formed upon the sleeve 180 which is placed upon balloon 181, the reservoirs may be alternatively formed directly upon the balloon surface rather than upon a separate sleeve 180.

In forming the reservoirs, several manufacturing methods such as micro machining, chemical etching, ablation (laser, ultrasound, RF, microwave, electron beam), selective laser sintering, etc., as well as various other polymer processing methods such as dip coating, injection molding, etc., can be utilized to create these reservoirs. Moreover, the geometries of the reservoirs may be designed in such a manner to provide for significant dose capacity, prevent premature release, and enable sufficient expansion in radial direction, thus effective drug release is achieved upon expansion of the balloon. This may be achieved, e.g., by forming the reservoirs 190 in a conical or angled configuration in the outer layer where each reservoir 190 may have a wider base adjacent to the balloon 181 surface and angle to a closed configuration as reservoir 190 extends radially away from balloon 181, as illustrated in the representative cross-sectional view of FIG. 13D. With balloon 181 in a deflated configuration, the apex of reservoirs 190 may be closed upon itself to contain the biological agent. However, as balloon 181 is expanded, the apex of reservoirs 190 may open to release the agents contained within.

Another variation is illustrated in the perspective view of FIG. 14A, which shows interconnected reservoirs 192 defined along the surface of balloon 181. The cross-sectional profile of FIG. 14B shows each reservoir 192 configured as a pore or well shape to which the agent may be added as a viscous fluid to facilitate its insertion and packing into the pores or reservoirs 192 of the outer surface. The thermal property of the viscous fluid is selected in a manner to cause significant reduction in the viscosity upon its exposure to the body temperature. This will further enhance drug transport into the tissue, when the balloon reaches its maximum diameter and brings the drug containing fluid in contact with the blood vessel.

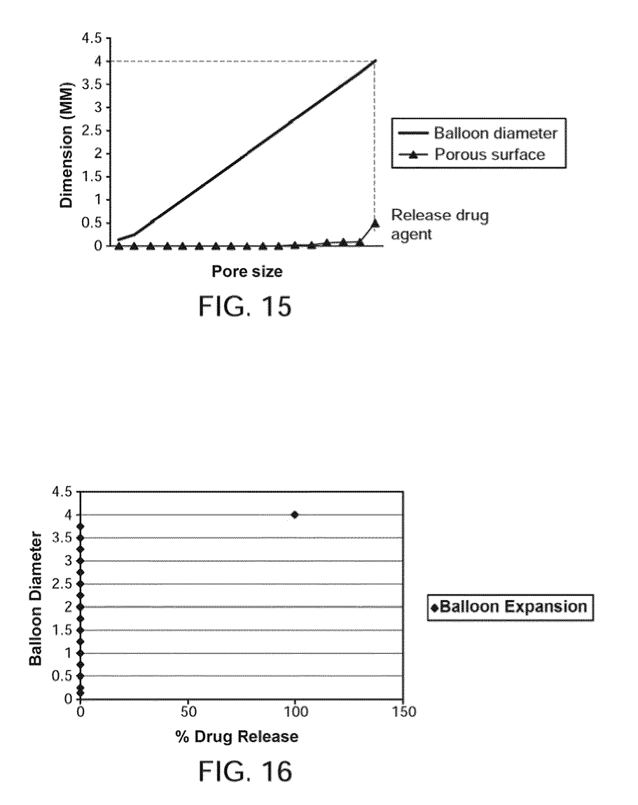

FIG. 15 is a graph showing an increase in pore size and correlated release of a drug agent when the balloon reaches its maximum diameter. This illustration is an example of a balloon diameter of, e.g., 4 mm, coupled with a porous surface with stretched pore size of, e.g., 0.5 mm.

FIG. 16 is a graph showing the complete release of a drug agent at a predetermined balloon diameter of, e.g., 4 mm.

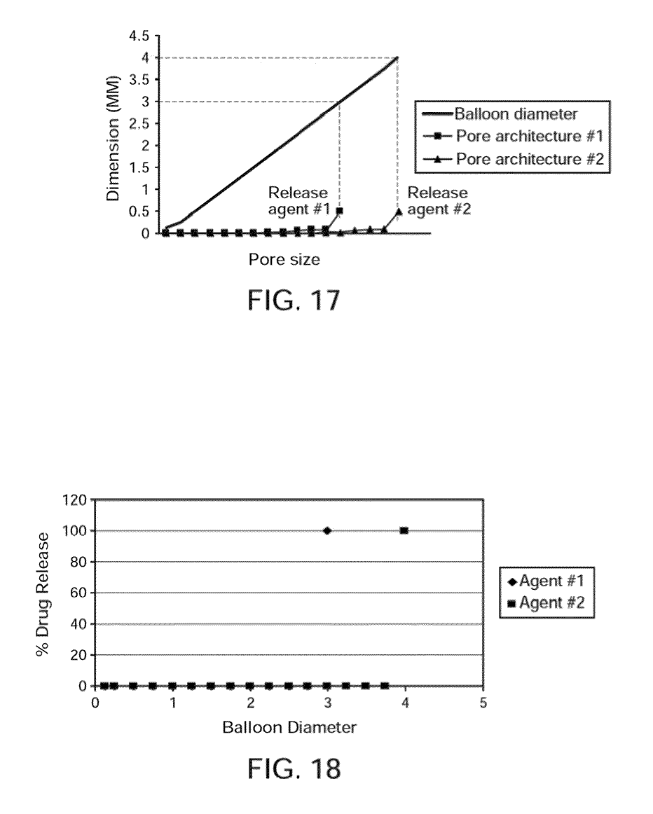

FIG. 17 is a graph showing an example of two different pore architectures responding to the balloon expansion. When the balloon reaches its first diameter of, e.g., 3 mm, pores of the first architecture open, causing the release of the first drug agent. Full inflation of the balloon to 4 mm diameter will open the pores of the second architecture, causing the release of the second drug agent.

FIG. 18 is a graph showing 100% release of a first drug agent when balloon researches its first diameter of, e.g., anywhere from 1 mm to 5 mm and particularly to 3 mm, followed by complete release of a second drug agent when the balloon is fully inflated to, e.g., anywhere from 5 mm to 10 mm and particularly to 4 mm in diameter.

Although various diameters for an inflatable balloon are described, these examples are illustrative of balloon inflation and an inflatable balloon as utilized herein may be inflated to any suitable diameter, e.g., 1 mm to 10 mm, for effecting a treatment.

In intravascularly advancing a balloon catheter having the porous outer layer disposed thereupon, an outer sheath may be used to cover the porous layer during delivery through the vasculature to retain any biologically active substances or agents placed, infused, or otherwise disposed within or upon the outer layer. However, the cross-sectional size of the sheath may undesirably increase the diameter of the balloon and porous outer layer, particularly for neurovascular applications where the vessels are tortuous and relatively small in diameter. Moreover, retraction of a sheath from the porous outer layer may be difficult depending upon the tortuous configuration of the delivery catheter. Furthermore, retracting the sheath may also undesirably remove some of the agent placed, infused, or disposed upon the porous outer layer. Delivery of the porous outer layer assembly without a sheath may also release undesirable amounts of the agent disposed within or upon the outer layer into the vasculature and any therapeutic amounts of agent upon the outer layer may also be diluted by the time the targeted tissue region is reached.

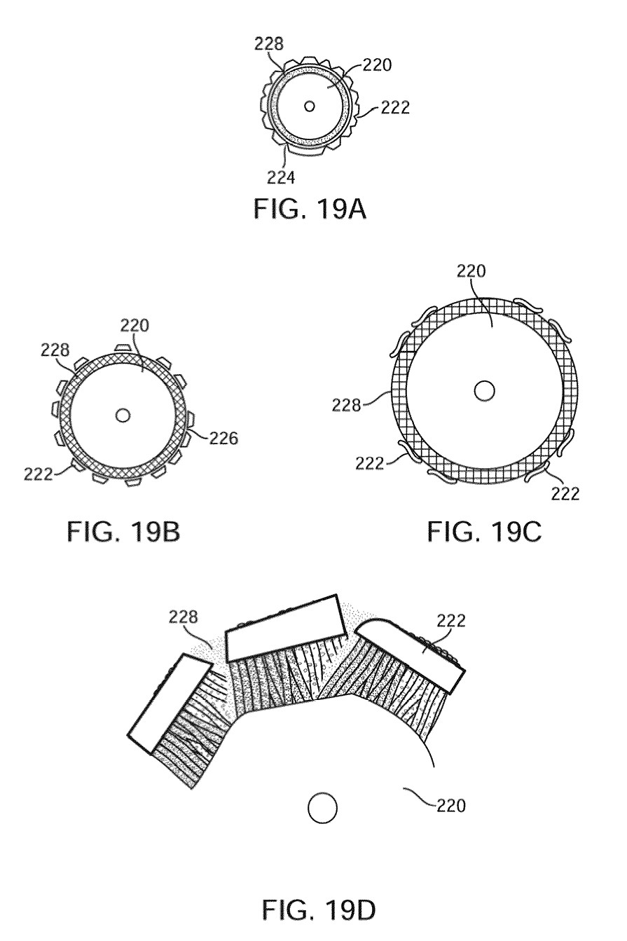

Accordingly, in one variation as shown in FIG. 19A, outer porous layer 228 disposed upon balloon 220 may be coated or otherwise encapsulated by a structurally jeopardized or weakened outer sheath 222. Outer sheath 222 may retain any biological agents placed or infused upon or within the outer layer 228 while maintaining a low-profile diameter of the assembly. The outer sheath 222 may be weakened by any number of mechanical discontinuities, e.g., using various techniques such as creating scores, notches, and/or cuts 224 along its surface so that when the balloon 220 is coupled with outer porous layer 228 and inflated, outer sheath 222 may be easily split or fragmented 226 along the weakened portions 224 of outer sheath 222 in a predictable manner due to the imparted radial stresses, as shown in FIG. 19B. Examples of materials which may be utilized for fabricating the outer sheath 222 may include, but not limited to, e.g., polysaccharides, hyaluronic acid (HA), alginates, PEG, PLA, PGA, PGA-PLA copolymers, or any of the other suitable materials described herein.

The balloon 220 and outer porous layer 228 may be further inflated and expanded, as shown in FIG. 19C, such that the outer sheath 222 is further stretched and ultimately disintegrated or decoupled from the porous layer 228. While the balloon 220 remains inflated, the biological agents entrapped in the cells of the stretched or otherwise deformed porous layer 228 may be exposed for contact with and delivery to a targeted tissue, as described above. FIG. 19D shows an enlarged cross-sectional view of the inflated balloon 220 coupled with porous layer 228 partially covered with remaining portions of disintegrated outer sheath 222. Utilizing a sheath 222 which disintegrates upon expansion of the balloon 220 eliminates complications relating to sheath retraction and also maintains a low-profile of the outer layer 228 as a thin layer of the outer sheath 222 may be used. Although the thickness of outer sheath 222 may be varied to suit different applications, the thickness may generally range anywhere from 1 μm to 500 μm.

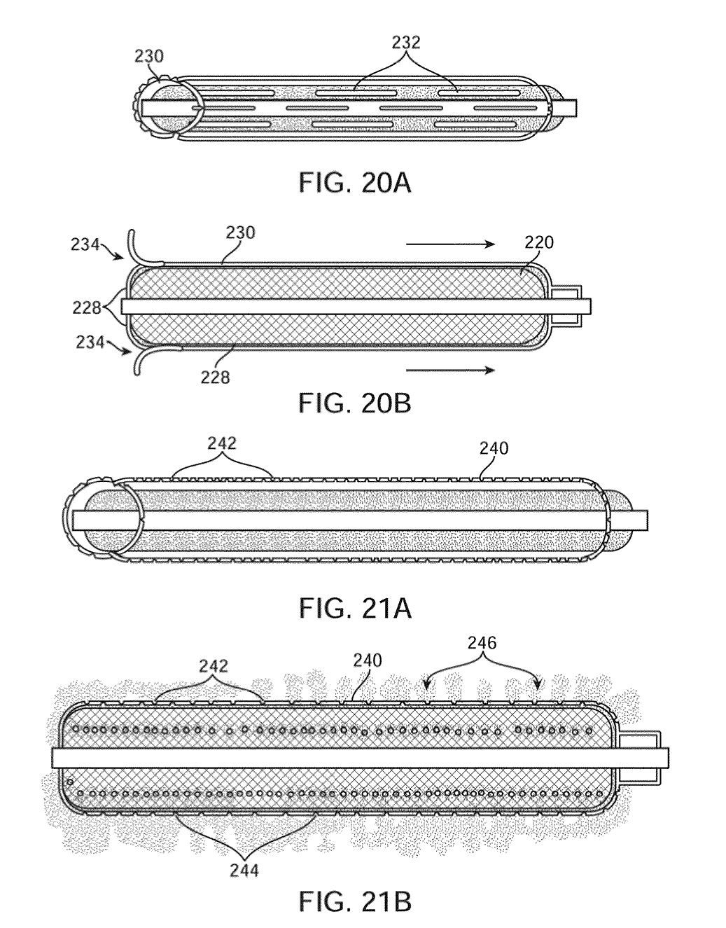

FIG. 20A shows another variation where the balloon may be covered with a structurally jeopardized outer sheath 230 where the sheath surface is weakened by multiple longitudinal grooves or cuts 232. Upon expansion of the balloon 220, outer sheath 230 may be unsheathed or ruptured due to the radial stresses imparted by the balloon 220. In the example of FIG. 20B, outer sheath 230 is illustrated rupturing initially at its distal end 234 to expose the underlying porous outer layer 228.

FIG. 21A shows a side view of yet another variation of a disintegrating outer sheath 240 which is structurally jeopardized by a plurality of perforations or holes 242 formed throughout the surface of outer sheath 240. The hole diameters may range individually or uniformly anywhere from 1 μm to 300 μm. As the balloon is inflated the perforations or holes 242 may become significantly increased in diameter 244 allowing the biological agent 246 to be released or available for treatment upon the targeted tissue, as shown in FIG. 21B. Alternatively, outer sheath 240 may begin to disintegrate along the perforations or holes 242 as the balloon is inflated to expose the underlying porous outer layer 228 for treatment.

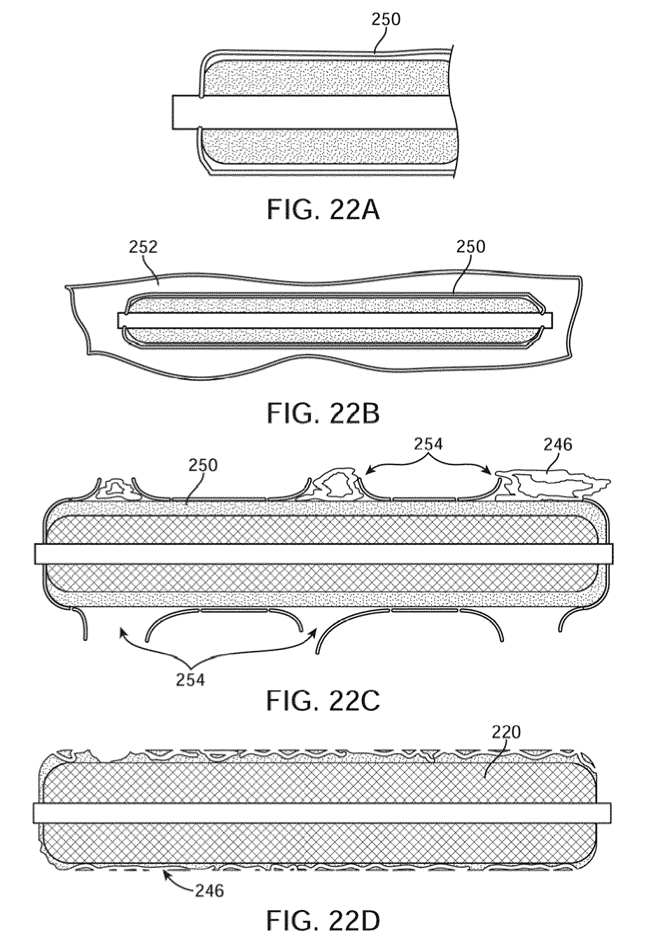

FIGS. 22A to 22D illustrate yet another variation where a thin layer of a structurally jeopardized polymeric biodegradable film 250 is placed on the outer surface of the porous outer layer to prevent any undesirable leakage of the biologically active substance coupled with the substrate. Biodegradable coating 250 can be formed a variety of the biopolymers such as, but not limited to, polysaccharides, hyaluronic acid (IIA), alginates, PEG, PLA, PGA, PGA-PLA co-polymers, starch, sucrose, fructose, chitosan, or any other suitable materials described herein, etc. As shown in FIG. 22B, when placed in the blood stream 252 the thin layer of biodegradable film 250 be dissolve and become completely disrupted upon full inflation of the balloon to create gaps or openings 254 along the film 250 and thus releasing biological agents 246 contained in the underlying porous outer layer, as shown in FIG. 22C. Disintegrated fragments of such a biocompatible and biodegradable film 250 will be easily dissolved in the blood stream and metabolized. Once the film 250 has been disintegrated or otherwise dissolved, the inflated balloon 220 and outer porous layer may remain to release the biological agents 246, as shown in FIG. 22D.

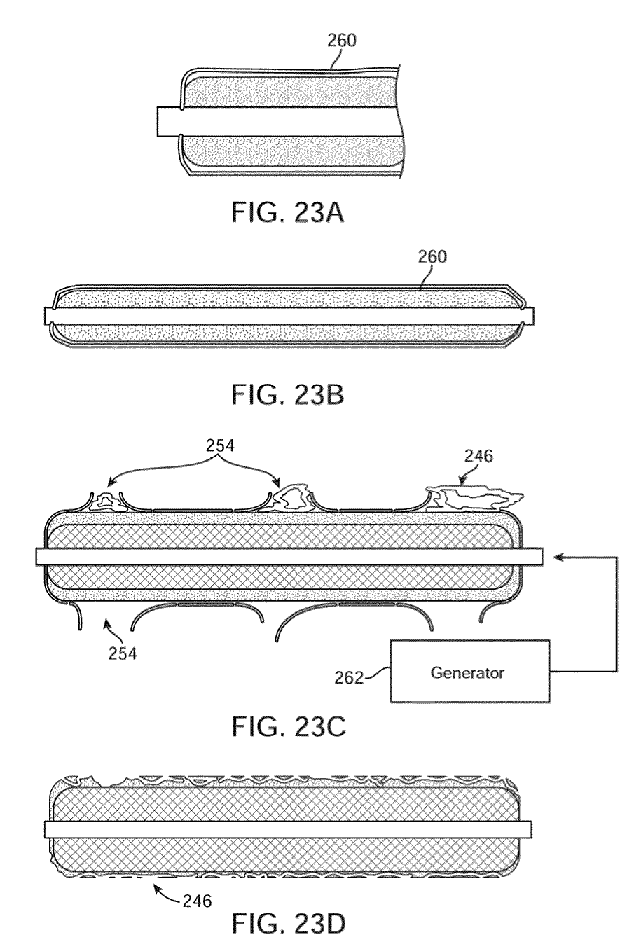

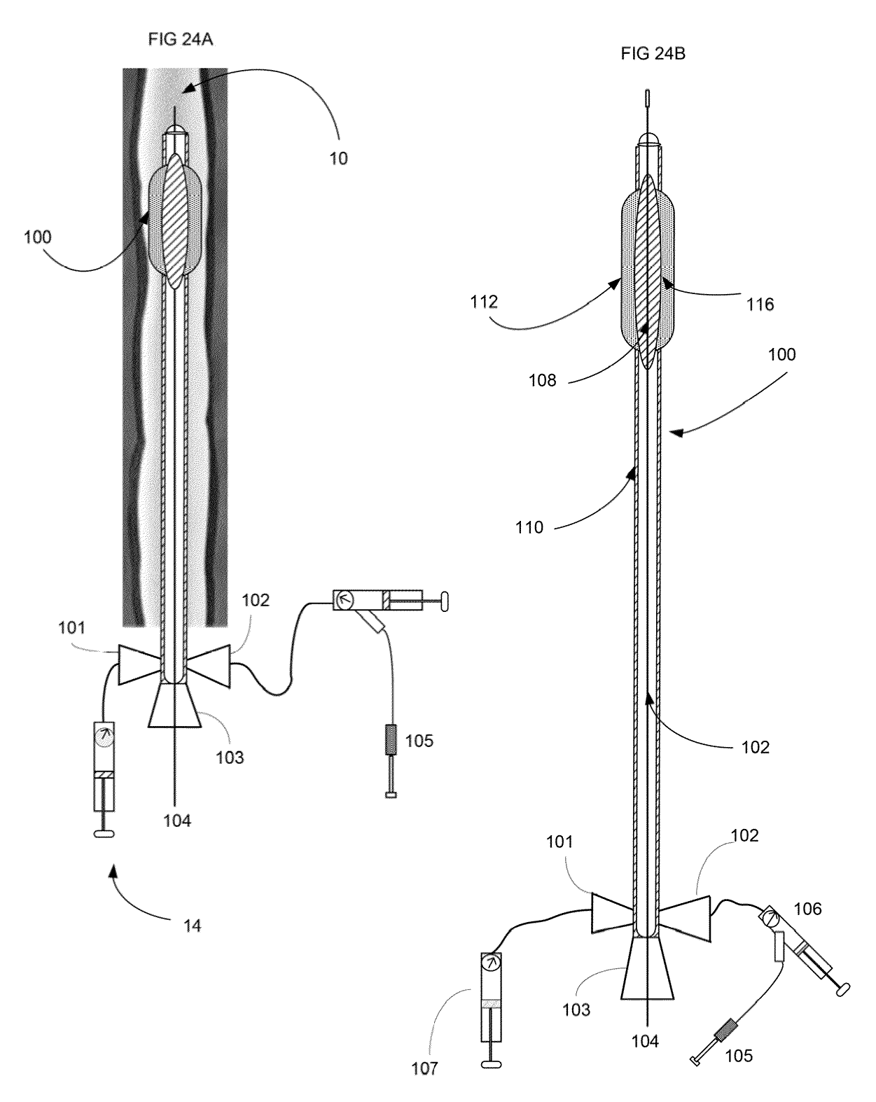

In yet another variation, the outer sheath may comprise a metallic erodable membrane 260 that may seal and/or encapsulate the porous outer layer and balloon assembly, as shown in FIGS. 23A and 23B. The metallic membrane 260 may be in electrical communication through the delivery catheter with a power supply, e.g., DC power generator 262, located externally of the patient body, as shown in FIG. 23C. Examples of suitable metallic materials which may be utilized as a membrane 260 may include, but are not limited to, e.g., Stainless steel, Magnesium alloys, NiTi alloys (Nickel-Titanium), Platinum, Platinum alloys, Gold, etc. The membrane 260 may be attached to a positive terminal while the patient is connected to a negative terminal of the DC power generator 262 such that when the balloon is expanded, a small amount of current may be applied to positively charge the metallic membrane 260 and negatively charge the patient. This electrical potential difference creates electrolysis between the membrane 260 and the patient, thereby causing positively charged metallic ions to move away from the membrane 260 and toward the blood stream. This erosion may cause unsealing 254 of the outer member 260 and release of the biological agent 246 for treatment upon the targeted tissue, as shown in FIGS. 23C and 23D.

Additionally and/or optionally, the metallic membrane 260 may be coupled with an additional drug or agent. During electrolysis and erosion of the membrane 260, metallic ions carrying the drug or agent may become eroded from membrane 260 and infused into the blood vessel for additional treatment upon the patient.

In a further embodiment, pharmaceutical agents such as Paclitaxol or other drugs may be incorporated into nanoparticles of positively charged polymers such as Chitosan. Alternatively, drug containing bioabsorbable polymers such as (D, L-Lactide-co Glycolide) PLGA or PLGA/PVA (Polyvinyl Alcohol) nanoparticles may be prepared. These particles are not intrinsically charged, but they may be coated with charged polymers such as Chitosan or other materials to form a composite structure. The nanoparticles may be coated within or upon the outer layer or alternatively, placed inside the silo/silos. When the electrical current is applied and the patient's body in negatively charged, the drug loaded nanoparticles with the opposite polarity will readily bind to the endothelial cells, thus enhancing intracellular transport of the drug.

In yet another variation, drug containing metallic nanoparticles such as Magnesium, Iron or their alloys may be coated or otherwise placed within or upon the outer layer. The outer layer is made of or coated with a metallic material to provide electro conductivity. Nanoparticles may be positively charged while the patient's body is negatively charged by using the power supply. Drug containing nanoparticles may be attracted to the negatively charged endothelial cells. This may increase efficiency of intracellular uptake of the drug.

In a further embodiment, the balloon may be constructed in such a manner to reduce the potential for short circuiting between the delivery system and arterial wall which are of two opposite polarities. Such a balloon may have the distal and proximal sections that are larger than the middle section. Only the middle section of the balloon may be conductive while the distal and proximal sections being none conductive. In application, the only large diameter sections will make contact with the vessel wall, while the middle conductive section is positioned at distance from the wall to prevent short circuiting. When energized, the charged nanoparticles may be attracted and bonded to the endothelial cells having an opposite polarity thereby enhancing the drug uptake efficiency.

Alternatively, rather than utilizing metallic materials for outer sheath 260, a thin layer of an electrically sensitive film made from a biodegradable coating can be formed out of bilipid membranes, peptides, and some polyelectrolytes. Such materials may change their structural properties under a DC current, RF energy, or ultrasound energy. These changes may be utilized to trigger the disruptions 254 of the coating film to thus release the drug or agent 246. Moreover, the sensitive film may be additionally and/or alternatively configured to be thermally or pit sensitive as well. Additional films may also include, e.g., proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans; e.g., hyaluronic acid; chitin chondroitin sulfate A, B, or C, keratin sulfate, keratosulfate and heparin; and activated polysaccharides such as dextran and starch derivatives.

Although devices and methods are described relative to a biologically active substance applied to the interior of the blood vessel device, it is to be understood that the other variations are not to be limited thereby. Indeed, other variations may be advantageously utilized for simultaneous angioplasty and anti-restenosis treatment of various blood vessels.

FIG. 24A illustrates an illustrative view of a blood vessel 10 of a patient body with one variation of the catheter treatment system 100 positioned therein. The catheter treatment system 100 may be introduced into the patient body via percutaneous access through the patient's skin and into the blood vessel 10 to be treated via guidewire inserted via guidewire port 104. The catheter system 100 may be advanced into the blood vessel 10 until the portion to be treated has been reached and/or traversed by the catheter system 100. As further shown in FIG. 24B, the catheter treatment system 100 may be connected via an inflation/deflation tubular member 12 to a pump 14 positioned externally of the patient.

Inflation and deflation of the balloon 108 is done by inflation pump 107 via inflation deflation port 101. Drug introduction is done using inflation pump 106 in conjunction with drug containing reservoir 105 via drug injection port 102.

Further examples of devices and methods which may be utilized herewith are described in further detail in U.S. patent application Ser. No. 11/852,711 filed Sep. 10, 2007 (U.S. Pat. Pub. 2008/0140002 A1), which has been incorporated herein by reference in its entirety above.

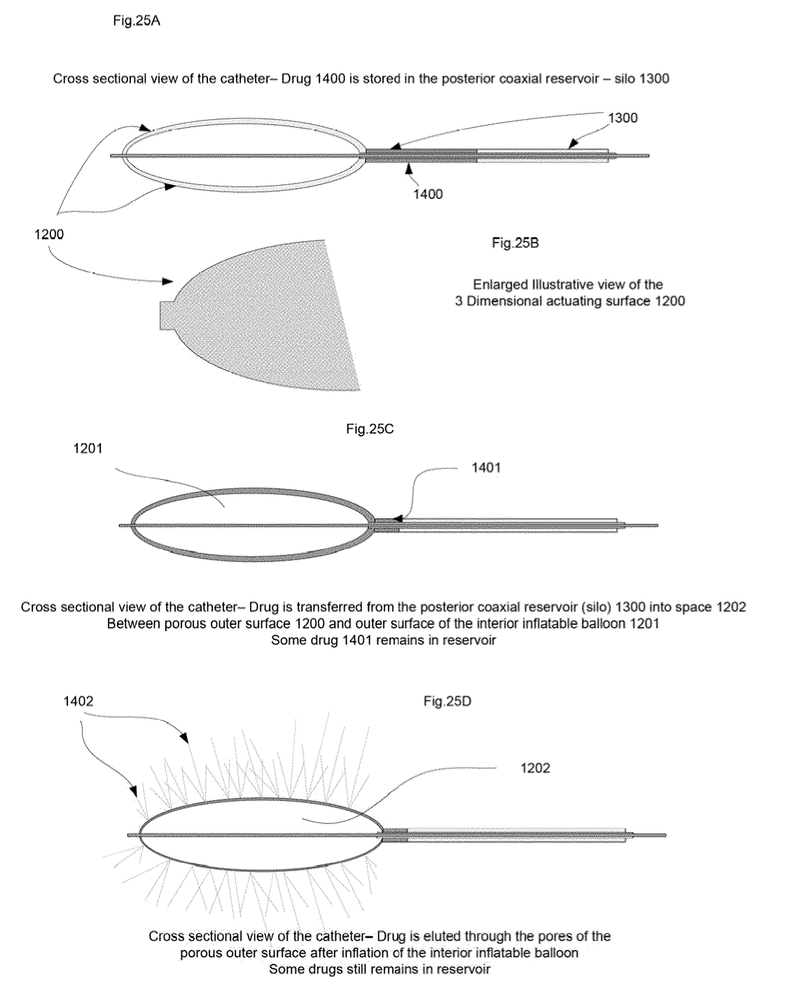

FIG. 25A shows a cross-sectional side view which shows a volume of one or more drugs located in a reservoir 1400, e.g., a silo, contained within the delivery system 1300. The silo is in fluid communication with the surface of the expandable actuating surface 1200 (e.g., 3-dimensional or 3D surface) and/or balloon. In application, the balloon is first expanded to a predetermined diameter, as shown in the side view of FIG. 25B. The biologically active agent is delivered to the 3D surface of the balloon, as shown in the cross-sectional side view of FIG. 25C, and subsequently released upon the tissue region of interest, as shown in FIG. 25D. The silo retains additional biologically active agent to allow for controlled release at a later time.

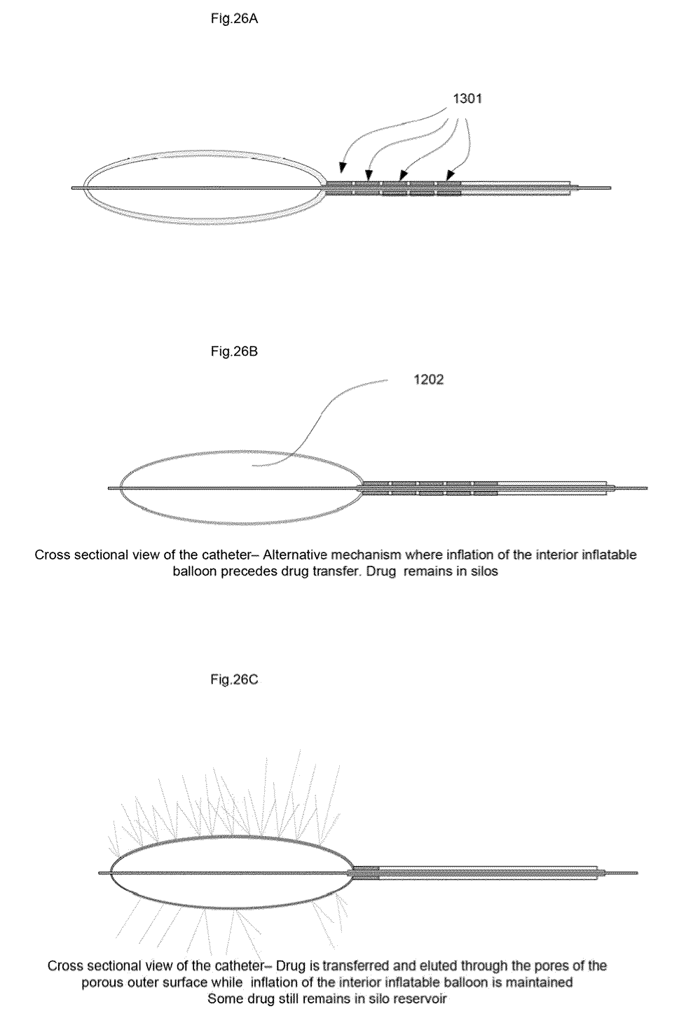

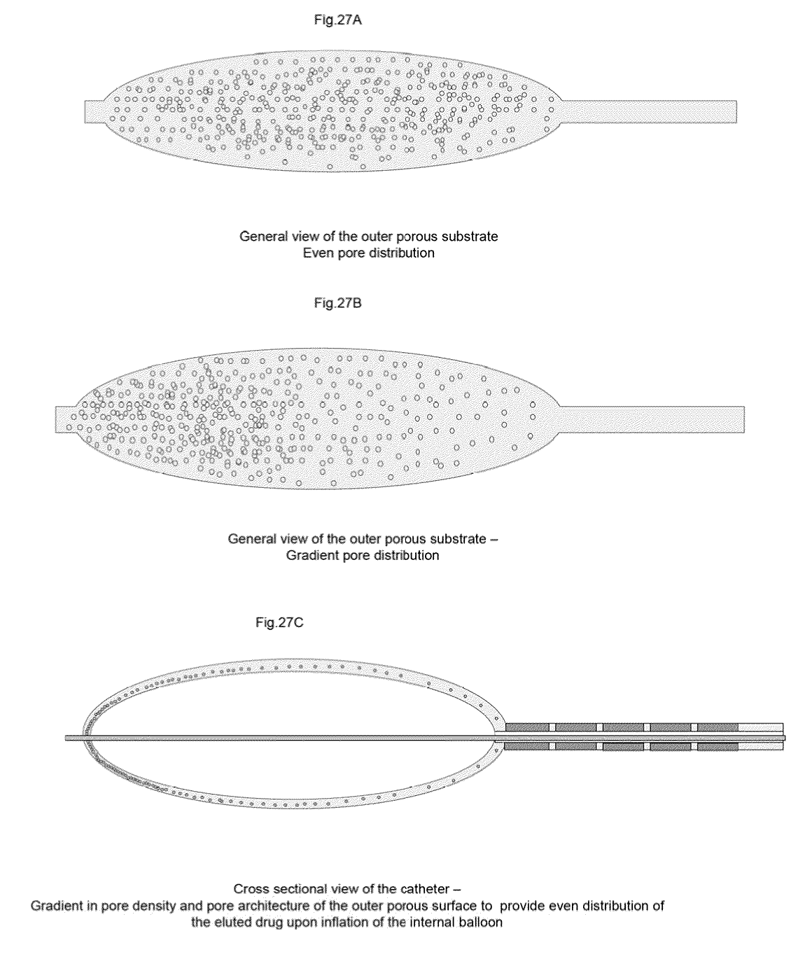

Yet another variation is shown in FIGS. 26A to 26C which illustrate multiple silos 1301 containing different biologically active agents, volumes or concentrations that are in fluid communication with the 3D balloon surface. Moreover, drug transfer from the one or more silos 1301 may be actuated prior to, during, or after balloon inflation and/or surface expansion. The gradient in pore density and/or architecture of the 3D surface is designed to provide an even distribution of the eluted drug upon inflation of the balloon. For example, as shown in FIG. 27A, the outer porous substrate positioned over the balloon may provide an even pore distribution for containing the one or more drugs distributed over the surface. Alternatively, the gradient pore distribution may be more dense near a distal end of the 3D surface relative to a proximal end of the surface, as shown in FIGS. 27B and 27C, where the gradient of pore distribution may be stepped or gradually increased or decreased from the distal end towards the proximal end of the 3D surface.

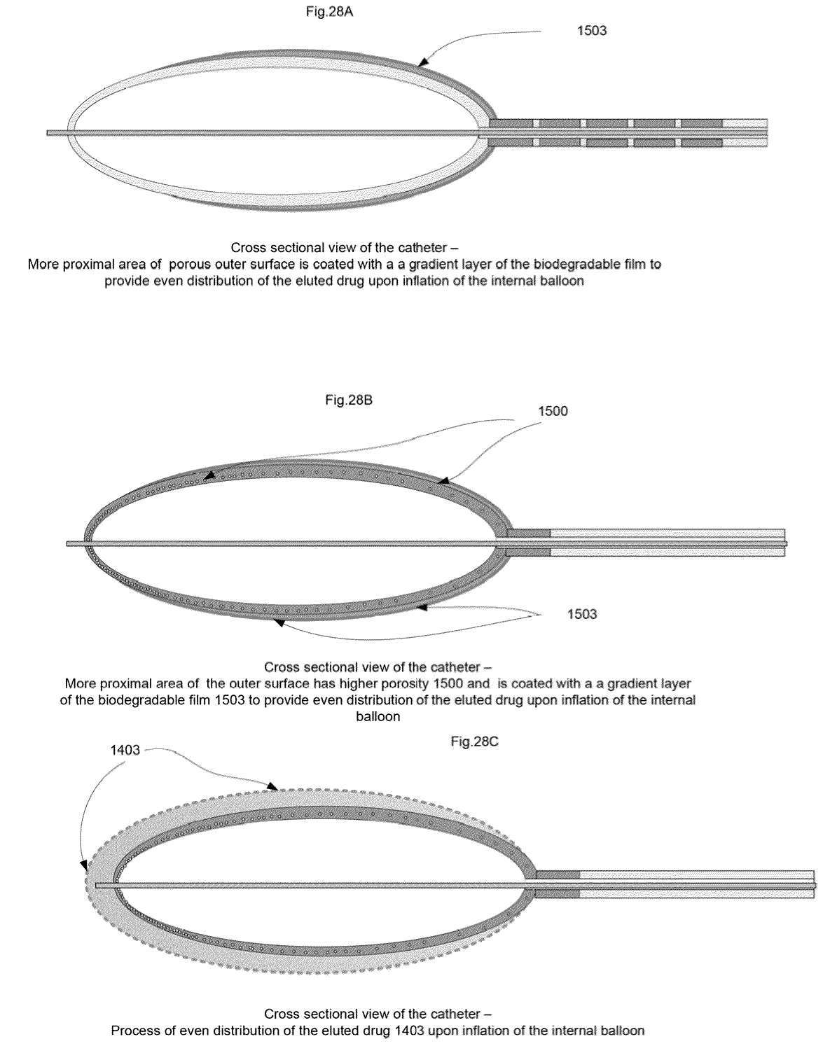

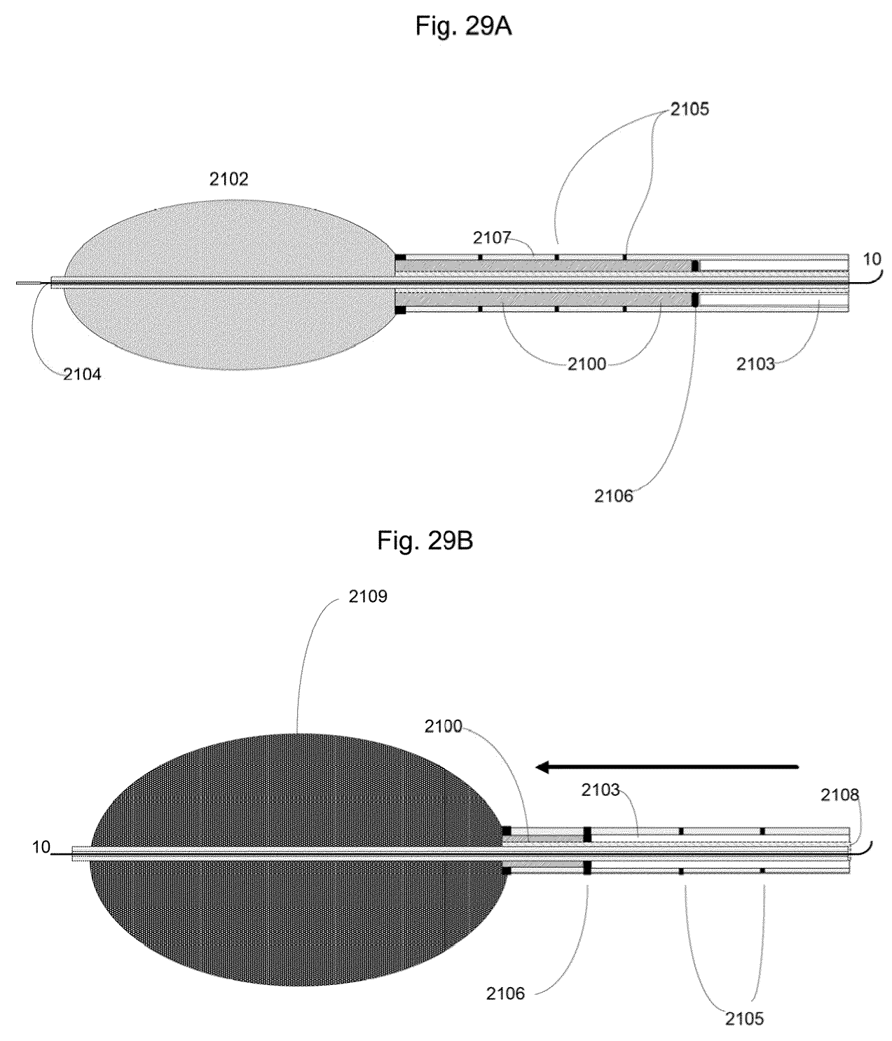

In yet another variation, as shown in the cross-sectional side view of FIG. 28A, at least a section of the 3D surface is coated with a biodegradable film to guide infusion of the biological substance to a preferred direction, allowing for an even distribution. For example, a coating or covering may be placed over a mid portion and/or proximal portion of the surface. When the one or more drugs are infused into a proximal portion of the surface, expansion of the balloon may be partially constrained by the coating or covering over the mid to proximal portion of the balloon, thereby forcing the one or more drugs to be infused into the remaining distal portion of the surface, as shown in FIGS. 28B and 28C.

Separation of individual silos may be accomplished by placement of one way valves between each volume of biological substance. These valves are directed towards the balloon and they will open upon application of the pressure, allowing transfer of biologically active substance to the 3D surface of the balloon. Another method of separation is the placement of an immiscible fluid between individual volumes of the biologically active substance which prevents the inter-mixing of adjacent individual volumes and potentially avoids the need for any additional structures for separating the individual volumes, as shown above in FIGS. 28B and 28C.

Yet another variation comprises having a rotational device at the proximal end of the delivery system. The device is designed to advance a plunger that is located at the proximal end of silos. Each rotation applies a force to the plunger pushing the biologically active substance through the silos. Predetermined number of rotations may deliver precise volumes of the biologically active substance to the 3D surface of the balloon. For example the rotating device can have several revolutions that correspond with delivery of a given dosage of the biologically active substance.

In a further embodiment, the boundaries of silos are defined by the radio opaque markers and similarly the plunger is made of or coated with such materials. In clinical application, the plunger is advanced forward and aligned with the silo's markers under fluoroscopic guidance. This alignment will ensure precise delivery of a given dose from the silo into the 3D balloon surface. This action may be repeated sequentially to treat multiple lesions.

Furthermore, the system may contain sufficient volume of biologically active substance to treat long or multiple lesions. In application, a precise dose of the biologically active substance may first be delivered by a predetermined number of revolutions of the rotating device treating a segment of the lesion. The system may then be moved to a new location of the lesion for subsequent treatment.

In additional embodiments, catheters may be graduated with radio opaque marker and plungers to visually control amount of injected drug, e.g., one drug or multiple doses.

FIG. 29A depicts a drug eluting delivery system comprising internal drug containing lumen 2101, partially filled with therapeutic agent 2100, inflatable balloon with “3D smart surface” 2102 (in deflated state), fluid for plunger operation 2103, radio opaque plunger 2106, and radio opaque graduation markers 2105. System is introduced by the guidewire 10, inserted via guidewire lumen 2104. Inflation and deflation of the balloon is done via lumen 2108.

FIG. 29B depicts a drug eluting delivery system in action where “3D smart surface” activated by inflation balloon into working state 2109, then pushing fluid 2103 and moving plunger 2106.

Drug 2100 is displaced and relocated into outer layer of the inflated balloon 2109 and then into surrounding target tissue. Radio opaque graduation markers 2105 allow for the direct control (via intra procedural fluoroscopy) of the amount of the displaced by the radio opaque plunger 2106, therapeutic agent 2100 and therefore direct control of the dosage delivered to target tissue. Assessment of the delivered amount is easy to calculate using registration of the position of the radio opaque plunger 2106 in respect with radio opaque graduation markers 2105.

The graduated catheter with one or more radio opaque markers and plunger may be used to visually control amount of injected drug, e.g., with different drugs or multiple doses.

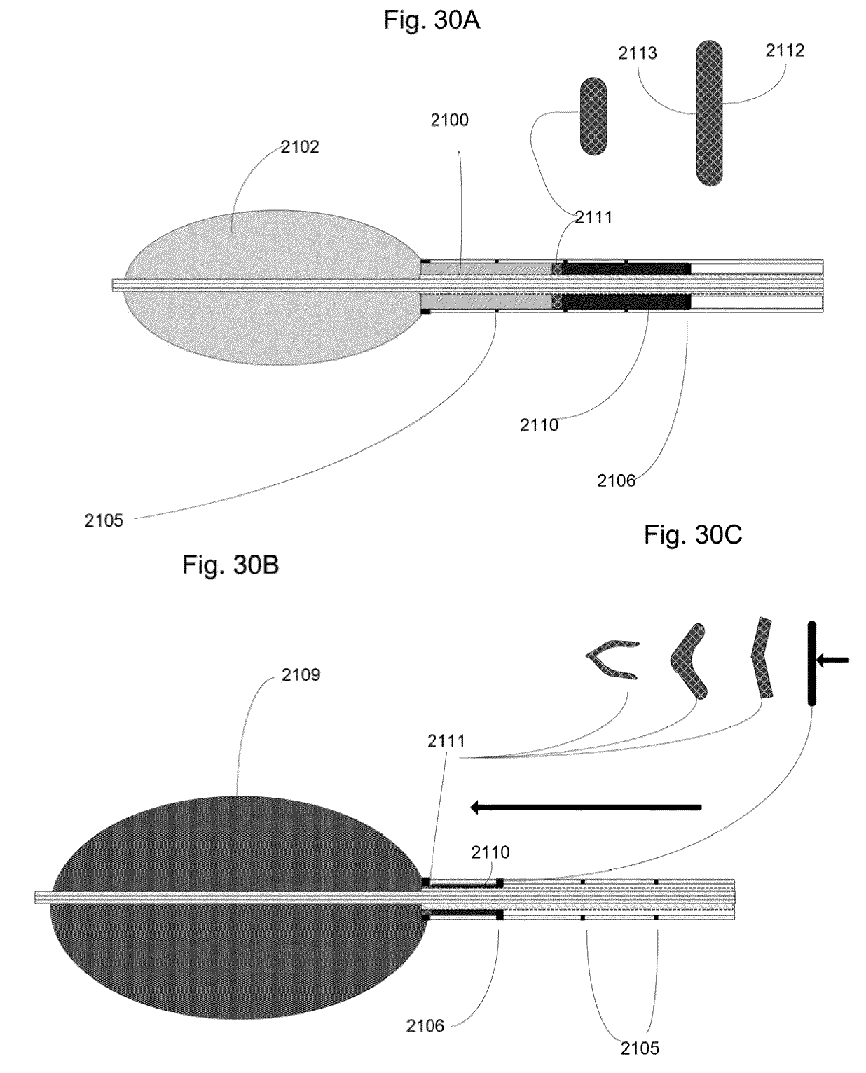

FIG. 30A depicts a drug eluting delivery system comprising internal lumen 2101, partially filled with different therapeutic agents; agent 2100 and agent 2110, inflatable balloon with “3D smart surface” 2102 (in deflated state), fluid for plunger operation 2103, radio opaque plunger 2106, and radio opaque graduation markers 2105. Various therapeutic agents 2100 and 2110 are separated via a solid collapsible partition (spacer) or a layer of the immiscible fluid 2111 such as liposome or emulsion. Liposomes can be composed of naturally-derived phospholipids with mixed lipid chains (like egg phosphatidylethanolamine), or of pure surfactant components like DOPE (dioleoylphosphatidylethanolamine). Emulsion can be composed of ethylene glycol, vinyl alcohol, hydrophilic acrylate, hydrophilic methacrylate, or hydrophilic amino acid subunits. Immiscible fluid 2111 allows to separate two or more different drugs. Immiscible fluid 2111 may contain a radio opaque component. That allows for the direct control (via intra procedural fluoroscopy) of the amount of the particular drug delivered to the target tissue.

FIG. 30B depicts a drug eluting delivery system in action where “3D smart surface” activated by inflation balloon into working state 2109, then pushing fluid 2103 and moving plunger 2106.

Drug 2100 is displaced and relocated into outer layer of the inflated balloon 2109 and then into surrounding target tissue. Drug 2110 is separated by the spacer 2111 and ready to be injected (Stand by mode).

FIG. 30C depicts a process of banding of the collapsible solid separator 2111 under hydraulic pressure exerted by the plunger 2106. One of the embodiments for the separator is porous collapsible sponge coated with hydrophilic 2112 and hydrophobic 2113 coatings. Sponge is composed in such manner that bends and then collapses under pressure and allows a stream of fluid move over it. Sponge can be composed of synthetic polymer such as PE, PP, silicone and polyurethane. It also can be composed of biopolymer such as gelatin, collagen, HA, Alginate.

Drug eluting delivery system containing a conductive mesh on the surface of the balloon to facilitate oriented movement of the charged drug containing particles and therefore enhance drug delivery to the vessel wall.

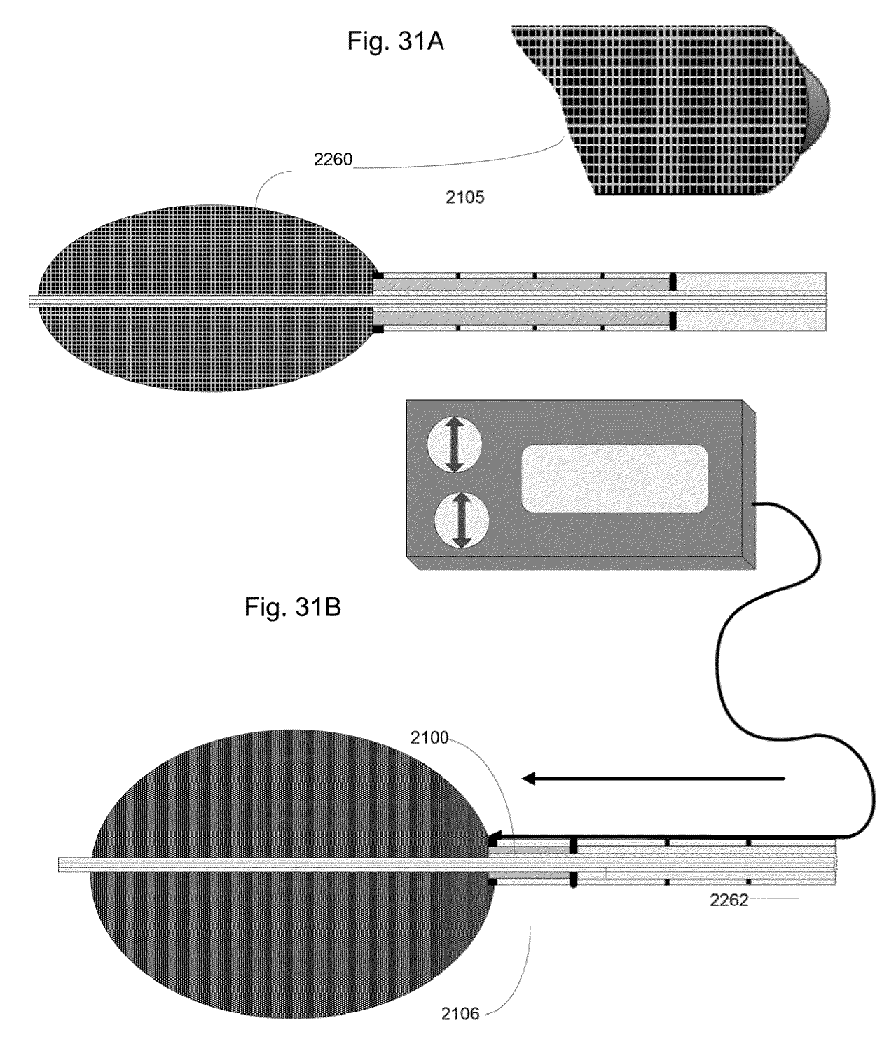

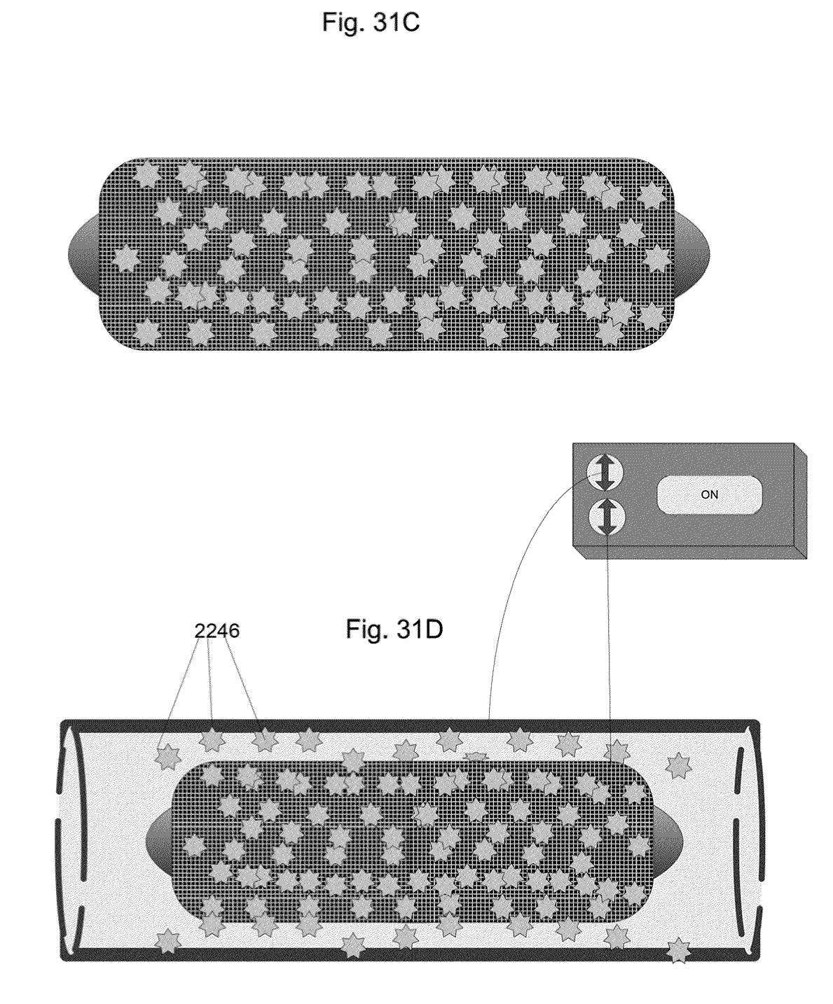

FIGS. 31A to 31D depicts an embodiment of the drug eluting delivery system comprising “3D surface”, silo drug reservoir and conductive mesh.

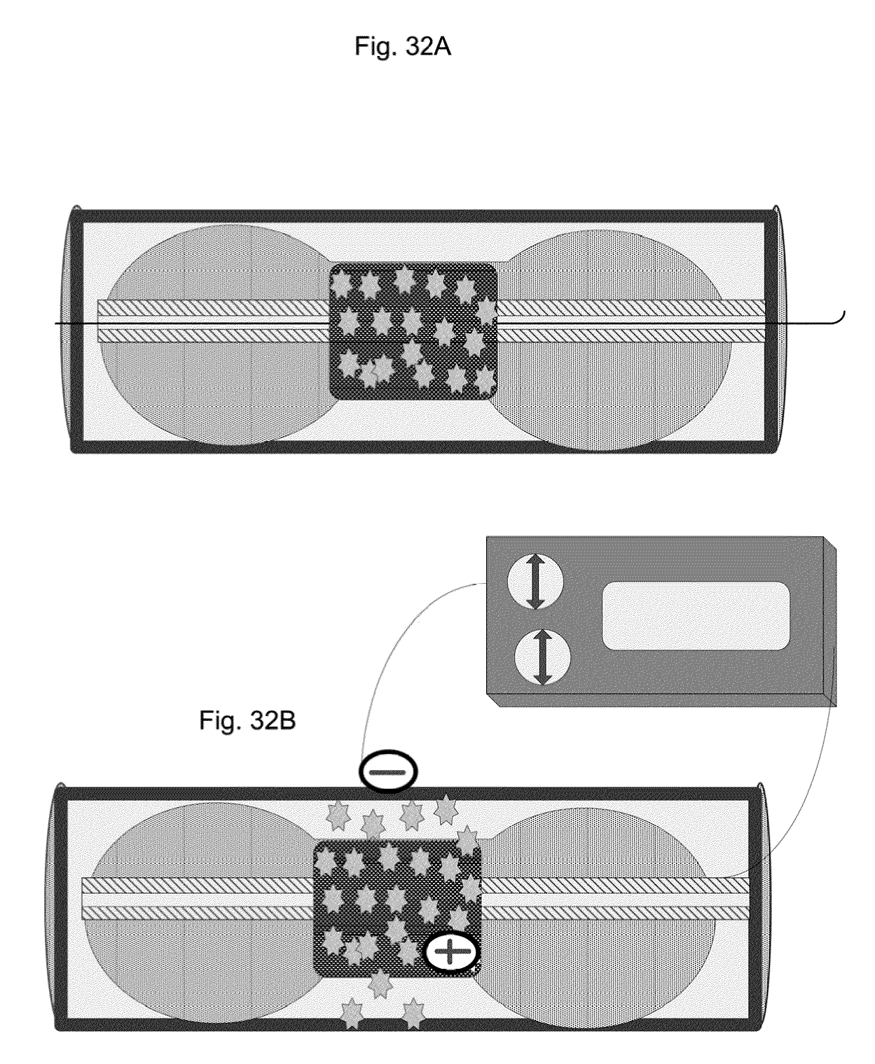

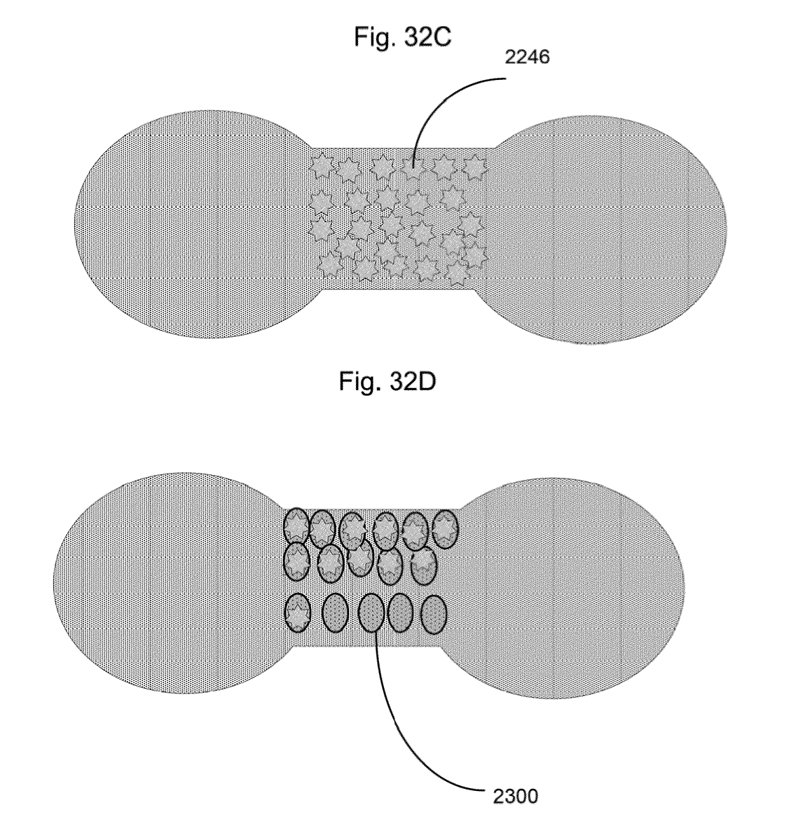

FIGS. 32A to 32D depicts an embodiment of the Drug eluting delivery system comprising “3D surface” and conductive mesh.

Electro active (charged) therapeutic agent containing compositions are also described. Both negatively and positively charged particles can be created via coupling of therapeutic agent (such as Paclitaxel) with various biomaterials.

Charged Composites containing therapeutic agents can be composed of Paclitaxel coupled with anionic polysaccharide, said polysaccharide being selected from the group consisting of carboxymethyl dextran, carboxymethyl amylose, carboxymethyl beta-cyclodextrin, dextran sulfate, cellulose sulfate, chondroitin, sulfate, heparin, heparan sulfate, dermatan sulfate, keratan sulfate and hyaluronic acid, or anionic (positively charged) polysaccaride such as chitosan.

Yet another embodiment for the Charged Composites containing therapeutic agents is—albumin-bound (Nab™) paclitaxel. Albumin is a versatile drug carrier in anti-cancer drug delivery system and it also has an actively targeting capacity to tumors. Certain product are commercially available and approved. For example nanoparticle albumin-bound (Nab™) paclitaxel (nab-paclitaxel; Abraxane®) has been approved in 2006 for use in patients with metastatic breast cancer who have failed in the combination chemotherapy, and so the nab-technology has attracted much interest in the anti-cancer drug delivery system. It comprises stable and negatively charged nanoparticles with size of approximately 0.1-0.2 μm.

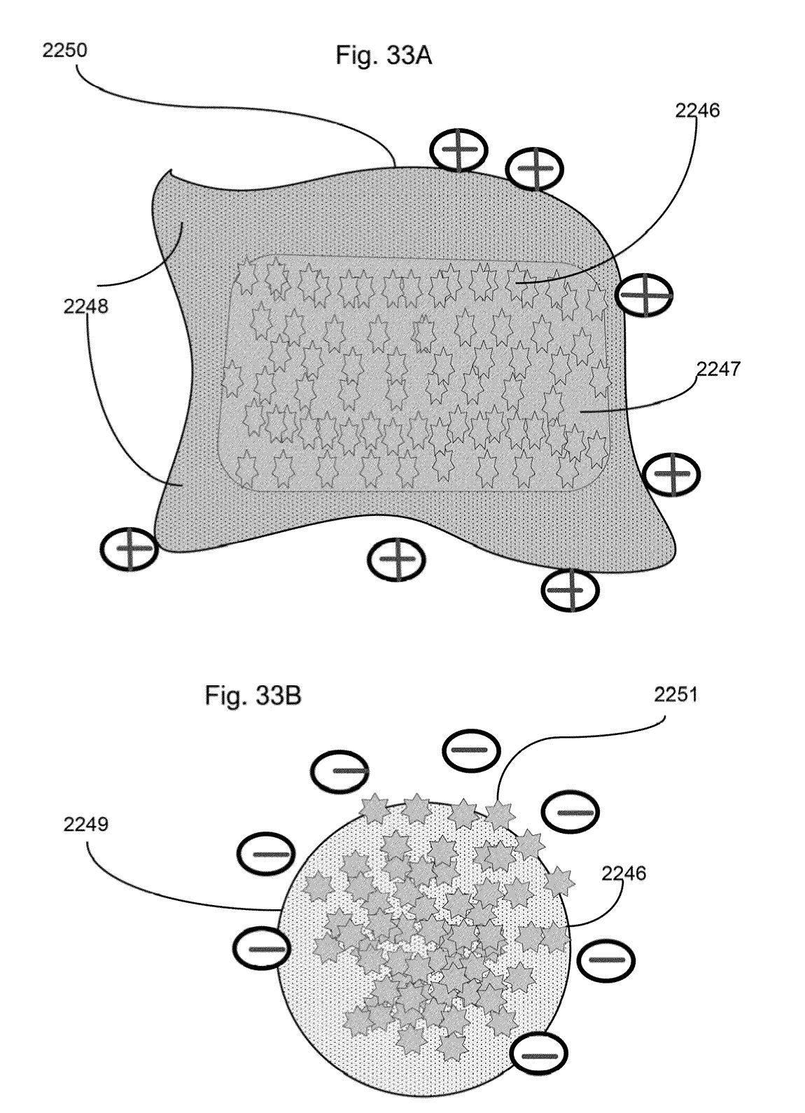

FIG. 33A depicts a composition of the positively charged particle 2250 comprising Paclitaxel 2246, dispersed in PGA-PLA copolymer 2247, encapsulated in the Chitosan matrix 2248.

FIG. 33B depicts a composition of the negatively charged particle 2251 comprising Paclitaxel 2246, coupled with Albumin 2249.

Typically, cancer treatment often relies on a multi-pronged approach with an initial surgical procedure followed by radiation and/or chemotherapy of the tissue surrounding the site of the surgery. Alternatively, local chemotherapy therapy may be carried out using a anticancer therapeutic source located outside the body in close proximity to the affected area.

In a further variation, any of the treatment devices and/or methods described herein may be utilized for the treatment of body cavities or lumens, e.g., surgically created cavities such as those formed for treatment of cancer. One the embodiments in particular is further directed treating tissue surrounding a surgically created resection cavity after surgical treatment of, e.g., malignant breast cancer. Generally, one of the described devices having a distal end of a catheter equipped with a modified 3D dynamic surface containing, e.g., non-conductive elements, conductive mesh, silo reservoir with a anticancer therapeutic agent, etc., may be inserted into the resection cavity to deploy an inflatable element at a desired location within the resection cavity in such a manner that, in this variation, non-conductive elements of the balloon surface may come into contact with inner surfaces of the resection cavity for treatment.

Internal chemotherapy therapy has several important advantages over other methods of treatment for breast cancer. For example, this procedure places the therapeutic agent such as Paclitaxel inside the cavity created by the removal of the tumor (i.e., the lumpectomy or resection cavity). This reduces the potential for side effects of the systemic treatment. In addition, the more targeted application of therapeutic agent permits application of stronger doses so that the treatment regime can be completed in a shorter time, often in a matter of days.

An exemplary method of delivering chemotherapy therapy may include a balloon catheter that is inserted into a tumor resection cavity created by the surgical removal of a tumor. Abraxane nanoparticle version of Paclitaxel developed by Abraxis BioScience can be utilized as a negatively charged particulate loaded into the silo reservoir of the catheter and then transported on demand onto the 3D surface of the balloon. Nonconductive elements of the balloon can be constructed using standard polymeric materials such as PU, PE and Silicone via dipping or injection molding. Conductive mesh made out of any conductive material such NiTi or Stainless Steel can be placed on the surface using melting, welding or co extrusion or inserted into the matrix during dipping. The mesh can be made in the form of the nanoparticle incorporated into the polymer surface using plasma discharge or polymeric surface ionizing techniques.

After the course of treatment has been completed, the balloon is deflated and is removed together with catheter. The chemo therapy delivered with the balloon catheter may be used alone, or may provide a very targeted boost to other types of therapy, such as external beam radiation therapy and/or systemic chemotherapy or brachytherapy.

Alternatively in order to prevent direct contact of the charged conductive surface with charged interior of the cavity, cavity can be filled with electrolyte such as normal saline. Balloon then will be expanded inside of the cavity to the predetermined volume that will exclude a possibility of such a contact.

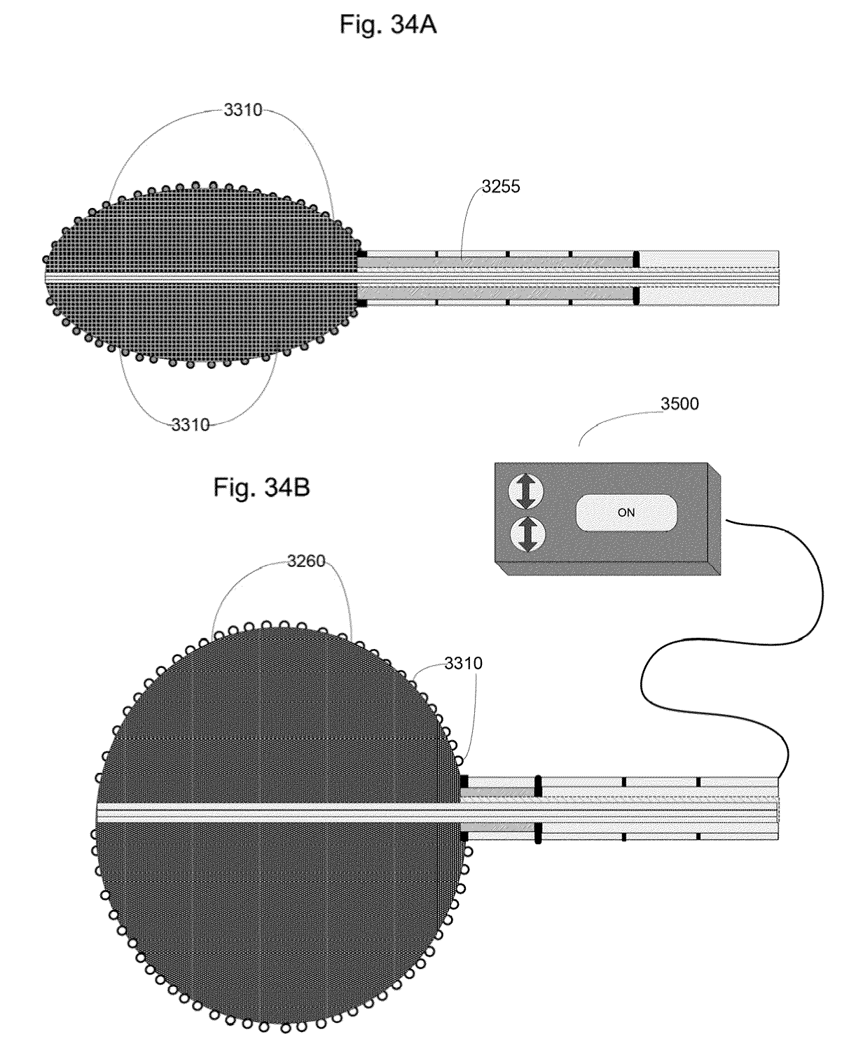

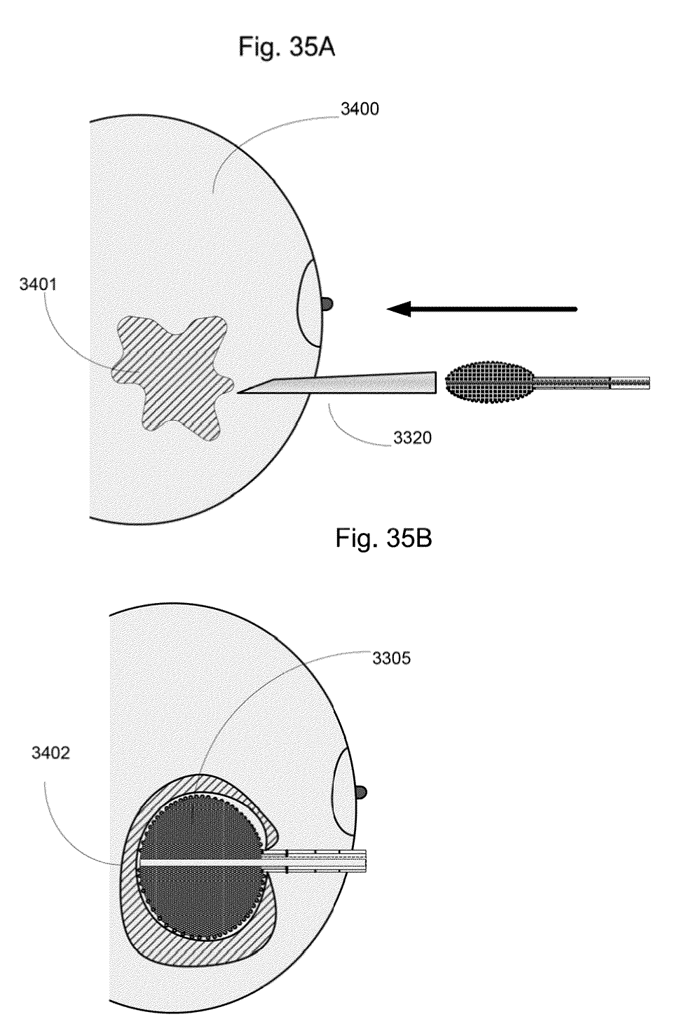

Turning now to particular illustrative examples, FIG. 34A and FIG. 34B show cross-sectional schematic diagrams illustrating an example of a modified 3D dynamic surface having one or more anticancer therapeutic agents 3255 stored in one or more silo reservoirs of the delivery system. The balloon of this variation may comprise a conductive mesh 3260 having a plurality of non-conductive elements 3310 providing isolation of the conductive zone from direct contact with a interior of the cavity once inflated into contact against the interior surfaces of the cavity to be treated.

FIGS. 35A and 35B illustrate cross-sectional schematic views showing an embodiment of how such a device may be delivered, placed, and expanded within a surgically-created cavity. A trocar introducer 3320 may be pierced into the tissue region, e.g., breast 3400, into alignment with the postsurgical cavity 3401. The treatment balloon 3305 may then be advanced through the trocar 3320, which may be subsequently removed from the tissue region, and into the cavity 3401 where it may be expanded until coming into contact against the interior surfaces of the cavity 3401 for direct treatment of the tissue region.

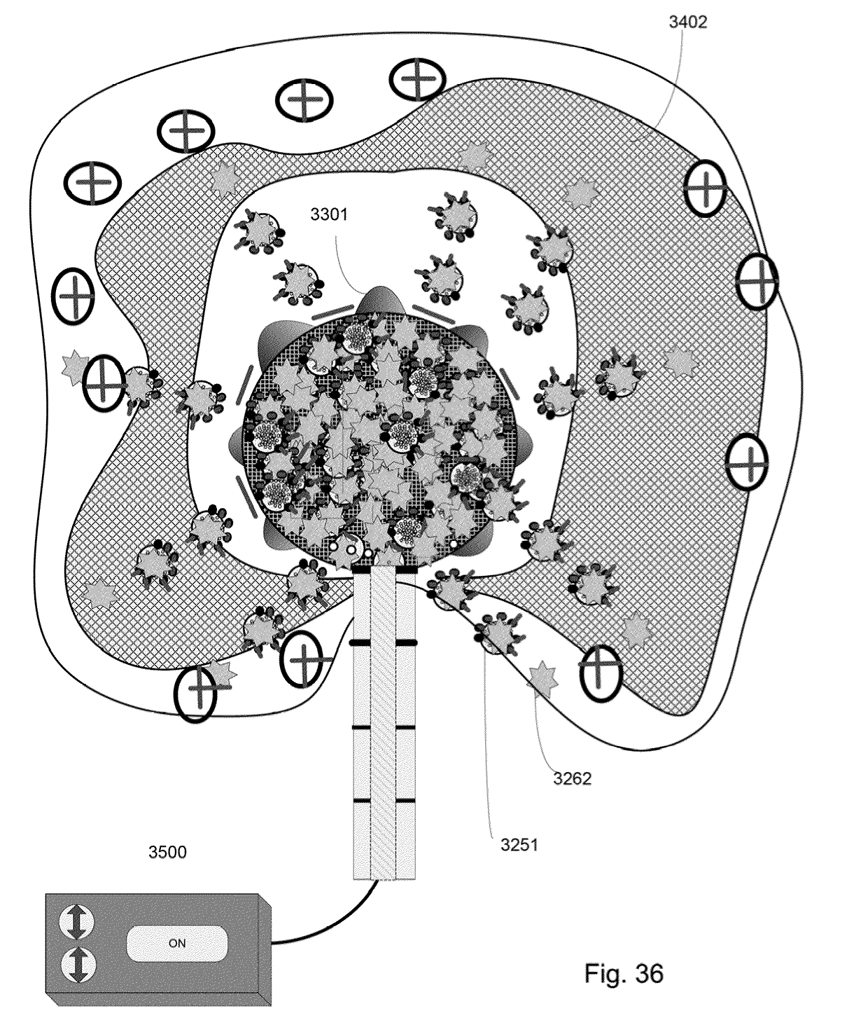

FIG. 36 illustrates a schematic diagram showing an enlarged view of expanded treatment balloon having a plurality of non-conductive elements 3301 for contact against the postsurgical cavity 3402. The surface of the cavity 3402 is positively charged using an electrical circuit generator 3500. Therapeutic agent 3262 coupled with a plurality of positively charged particles 3251 is transported from the silo reservoir into the 3D surface 3310 of the balloon containing the conductive mesh 3260. Driven by the current generated by the generator 3500, charged particles may migrate to the tissue of the cavity where drug 3262 is then released and absorbed the tissue. Non-conductive elements 3301 prevent contact of the mesh 3260 with the positively charged surface of the cavity 3402.

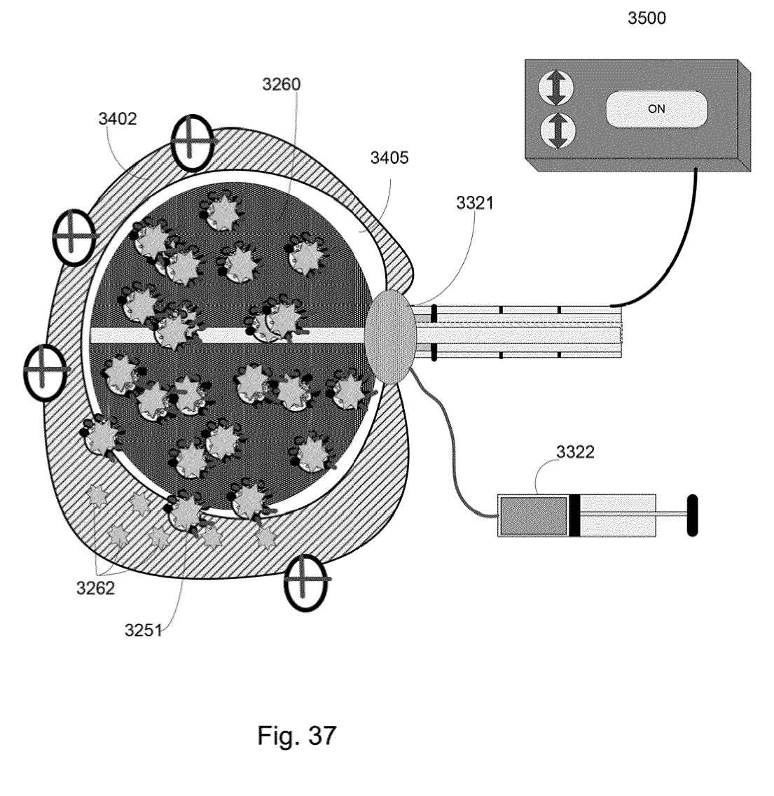

FIG. 37 is a schematic diagram showing an enlarged view of an expanded treatment balloon without the non-conductive elements on the surface of the balloon. Instead an electrolyte such as normal saline 3405 is introduced into the expanded postsurgical cavity 3402. Treatment balloon is inserted into the saline filled cavity and secured in position relative to the tissue using, e.g., an inflatable plug 3321 which may be hydraulically operated with a syringe or inflation deflation pump 3322. The surface of the cavity 3402 is positively charged using an electrical circuit generator 3500. One or more therapeutic agents 3262 coupled with a plurality of positively charged particles 3251 is transported from the silo reservoir into the 3D surface 3310 of the balloon containing the conductive mesh 3260. Driven by the current generated by the generator 3500, these charged particles may migrate to the tissue of the cavity 3402. One or more drugs 3262 may be then released and absorbed into the tissue. The saline layer 3405 between the conductive mesh and inner surface of the cavity may prevent contact of the mesh 3260 with the positively charged surface of the cavity 3402 and enable the migration of the charged particles.

In yet another variation for application of the devices and methods described herein is use for intra-uterus and/or intra-vaginal local treatment, such as local chemotherapy or local application of any therapeutic agent, e.g., hormones, steroids, and many other agents for direct application to the tissue. Many gynecological pathological conditions require medical procedures and maintenance of a uteral and/or vaginal cavity within a patient's body. For example, the treatment of certain tumors such as fibroids and cysts may require a multi-faceted approach that includes a combination of surgery, radiation therapy and chemotherapy. In such an approach, after an initial surgical procedure has been performed to remove as much of a tumor as possible, radiation and chemotherapy are performed to kill remaining cancerous cells that could not be removed surgically. Alternatively, the devices and methods may be used as a stand-alone treatment, e.g., for local chemotherapy applications such steroid or hormonal infusion. These pathological conditions can also be treated by inserting therapeutic materials directly into the cavity, in close contact with the affected tissues. For instance, the cavity 3402 illustrated in FIG. 37 may represent a diseased uterus having a device with a 3D surface and therapeutic agent, such as an anticancer agent, stored in the silo reservoir, for infusion into the uterus.

The applications of the devices and methods discussed above are not limited to the treatments outlined in this application but may include any number of further treatment applications. Modification of the above-described assemblies and methods for carrying out the invention as well as combinations of various features between examples, and variations of aspects of the invention that are obvious to those of skill in the art are intended to be within the scope of this patent.

What is claimed is:

1. An apparatus having a controlled delivery of one or more biologically active substances against or upon a tissue region of interest. comprising:
a catheter having an inflatable balloon:
an outer layer at least partially covering the balloon: and
at least one biologically active substance placed within or upon the outer layer.
wherein expansion of the balloon to a first diameter releases the at least one biologically active substance from the outer layer in a controlled manner for application against or upon the tissue region of interest while the outer layer retains additional biologically active substances within the layer in a controlled manner to allow for controlled release at a later time.

2. An apparatus having a controlled delivery of one or more biologically active substances against or upon a tissue region of interest. comprising:
a catheter having an inflatable balloon:
an outer layer at least partially covering the balloon: and
at least one reservoir, e.g., a silo located proximal to the inflatable balloon;
at least one biologically active substance placed within or upon the silo,
wherein the silo is in fluid communication with the outer layer,
wherein first inflation of the balloon expands the target lesion without release of the biological substance, and
wherein second inflation of the balloon releases at least one biologically active substance from the outer layer in a controlled manner for application against or upon the tissue region of interest while the silo retains additional biologically active substances in a controlled manner to allow for controlled release at a later time.

3. The apparatus of claim 2 wherein the outer layer is in fluid communication with the silos containing at least one biologically active substance and outer layer is filled with first biologically active substance from the first silo,
wherein inflation of the balloon to the first diameter releases a first biologically active substance from the first silo,
wherein outer layer is filled with second biologically active substance from the second silo,
wherein further inflation of the balloon to a second diameter releases the second biologically active substance from the second silo, and
wherein the second biologically active substance is retained within the second silo until the second diameter is obtained.

4. The apparatus of claim 2 wherein the catheter comprises an elongate flexible member having the inflatable balloon positioned near or at a distal end of the member.

5. The apparatus of claim 2 wherein the outer layer comprises a material for absorbing and retaining the at least one biologically active substance.

6. The apparatus of claim 5 wherein the material has a first state where the biologically active substance is retained within reservoirs which are at least partially closed and a second state when the balloon is inflated where the biologically active substance is released from opened reservoirs.

7. The apparatus of claim 5 wherein the material has a first state where the balloon expands without the biologically active substance and a second state where the biologically active substance is retained within reservoirs which are at least partially closed and a third state when the balloon is inflated where the biologically active substance is released from opened reservoirs.

8. The apparatus of claim 6 wherein the biologically active substance is released in the second state when the balloon has an inflated diameter of at least 1 mm to 10 mm.
